(19) United States
(12) Patent Application Publication
(10) Pub. No.: US 2010/0168120 A1
(43) Pub. Date: Jul. 1, 2010

(54) SALTS OF PYRIDAZINE COMPOUNDS

(75) Inventors: D.Martin Watterson, Chicago, IL (US); Linda Van Eldik, Chicago, IL (US); Wenhui Hu, Guangzhou Science Park (CN)

Correspondence Address: MILLEN, WHITE, ZELANO & BRANIGAN, P.C., 2200 CLARENDON BLVD., SUITE 1400, ARLINGTON, VA, 22201, US

(73) Assignee: NEUROMEDIX INC., Toronto, ON (CA)

(21) Appl. No.: 12/298,624

(22) PCT Filed: Apr. 27, 2007
(86) PCT No.: PCT/US2007/010368

§ 371 Date: Mar. 17, 2010

Related U.S. Application Data
(60) Provisional application No. 60/796,085, filed on Apr. 28, 2006.

(51) Int. Cl. A61K 31/506; C07D 403/14; A61P 29/00

(52) U.S. Cl. 514/252.02; 544/238

(57) ABSTRACT

The invention relates to stable and substantially purified synthetic pharmaceutically acceptable acid addition salts of pyridazine compounds of the formula I wherein R10 is hydrogen, hydroxyl, alkyl, alkoxy, alkenyl, alkynyl, alkylene, aryl, heteroaryl, sulfonyl, sulfinyl, sulfenyl, amino, imino, azido, thiol, thioalkyl, thioalkoxy, thioaryl, nitro, cyano, halo, =0, ═S, carboxyl, carbonyl, carbamoyl, carboxamide, or phosphonate, and R11 is alkyl, alkoxy, alkenyl, alkynyl, alkylene, alkenylene, alkenyloxy, aryl, aryloxy, arylalkoxy, aroyl, heteroaryl, heterocyclic, acyl, acyloxy, sulfonyl, sulfinyl, sulfenyl, amino, imino, azido, thiol, thioalkyl, thioalkoxy, thioaryl, nitro, cyano, halo, =0, ═S, phosphonate, carboxyl, carbonyl, carbamoyl, carboxamide, or ureido. The invention also relates to formulations, dosage forms and compositions comprising the salts, and methods of using the salts, formulations, dosage forms and compositions.

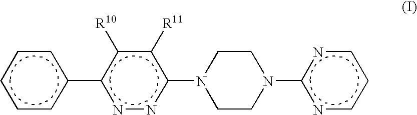

### FIELD OF INVENTION

The invention relates to certain salts of pyridazine compounds, compositions comprising the salts, and methods of using the salts and compositions.

### BACKGROUND OF INVENTION

Appreciation of the role played by glia activation in neurological disorder pathogenesis has increased rapidly in the past decade. Excessive glial activation is now known to be an early and key contributor to the progression of pathophysiology in a variety of neurological disorders, such as Alzheimer's disease (AD), traumatic brain injury (TBI), and neuropathic pain (Craft, J. M.; et al, Expert Opin. Ther. Targets 2005, 9, 887-900; Rothwell, N. Brain Behave. Immun. 2003, 17, 152-157; Warkins, L. R., Nature Rev. Drug Disc. 2003, 2, 973-985). Up-regulation of potentially damaging glia activation products, such as proinflammatory Cytokines, may also underlie the enhanced susceptibility to neurological dysfunction seen after a prior injury. For example, there is an enhanced risk of dementia and increased severity of AD-like pathology after a prior head injury (Mrak, R. E. et al., Neurobiol. Aging 2005, 26, 349-354; Guo, Z. et al, Neurology 2000, 54, 1316-1323), and many TBI patients subsequently develop a seizure disorder that is poorly responsive to conventional anti-epileptic drugs (D′Ambrosio, R. and E. Perucca, Curr. Opin. Neurol. 2004, 17, 7431-735). In addition, morphine-induced elevations in proinflammatory cytokines can cause acute opioid tolerance and withdrawal-induced pain enhancement after chronic morphine (Watkins, L. R. et al., Trends Neurosci. 2005, 28, 661-669). Therefore, the development of therapeutics that suppress elevations in glial proinflammatory cytokines could have broad impact across a number of neurological disease indications. Pyridazine compounds have been identified as potential selective inhibitors of glial activation pathways (US Published Application No. 2003-0176437, PCT Published Application No. WO 03/018563; PCT Application No. PCT/US05/39541).

### SUMMARY OF INVENTION

The present invention relates to stable and substantially purified synthetic pharmaceutically acceptable acid addition salts of pyridazine compounds of the formula I:

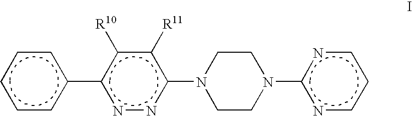

wherein is R¹⁰ is hydrogen, hydroxyl, alkyl, alkoxy, alkenyl, alkynyl, alkylene, aryl, heteroaryl, sulfonyl, sulfinyl, sulfenyl, amino, imino, azido, thiol, thioalkyl, thioalkoxy, thioaryl, nitro, cyano, halo, ═O, ═S, carboxyl, carbonyl, carbamoyl, carboxamide, or phosphonate, and R¹¹ is alkyl, alkoxy, alkenyl, alkynyl, alkylene, alkenylene, alkenyloxy, aryl, aryloxy, arylalkoxy, aroyl, cycloalkyl, cycloalkenyl, cycloalkynyl, cycloalkoxy, heteroaryl, heterocyclic, acyl, acyloxy, sulfonyl, sulfinyl, sulfenyl, amino, imino, azido, thiol, thioalkyl, thioalkoxy, thioaryl, nitro, cyano, halo, silyl, silyloxy, silylthio, ═O, ═S, phosphonate, carboxyl, carbonyl, carbamoyl, carboxamide, or ureido.

In aspects of the invention, R¹⁰ is hydrogen, alkyl, alkoxy, aryl, amino, thiol, halo, ═O, ═S, phosphonate, or carboxyl, more particularly R¹¹ is hydrogen or alkyl.

In other aspects of the invention, R¹¹ is alkyl, alkoxy, alkenyl, aryl, or heteroaryl, more particularly alkyl or heteroaryl.

Pharmaceutically acceptable acid addition salts of pyridazine compounds of the formula I according to the invention may be substantially different from the parent or lead compounds (e.g. 4,6-diphenyl-3-(4-pyrimidin-2-ylpiperazin-1-yl)pyridazine) in activity or physical properties, in addition to being structurally and physically different therefrom. The salts may also provide pyridazine compounds of the formula I and formulations comprising same with enhanced biological, chemical and/or manufacturing properties.

Pharmaceutically acceptable acid addition salts of pyridazine compounds of the formula I may be used to prepare formulations, dosage forms or pharmaceutical compositions. Therefore, the invention provides a method for preparing a formulation, dosage form or pharmaceutical composition comprising mixing an acid addition salt of a compound of the formula I according to the invention, preferably a halide salt, more preferably a chloride salt of a compound of the formula I, into a selected pharmaceutical vehicle, excipient or diluent, and optionally adding other therapeutic agents.

The invention also contemplates a formulation, dosage form, or pharmaceutical composition, in particular an improved formulation, dosage form, or pharmaceutical composition, comprising a pharmaceutically acceptable acid addition salt of a compound of the formula I, preferably a chloride or bromide salt.

A salt of this invention or formulation, dosage form or composition comprising same may be characterized by enhanced solubility and decreased molecular weight compared with a parent or lead compound (e.g. 4,6-diphenyl-3-(4-pyrimidin-2-ylpiperazin-1-yl)pyridazine) while retaining in vivo functions.

A salt or formulation, dosage form or composition according to this invention may be further characterized by one or more of the following: (a) favorable stability; (b) favorable bioavailability; (c) favorable hygroscopicity; and/or (d) substantially similar or lower toxicity relative to the parent or lead pyrizadine compound (e.g. 4,6-diphenyl-3-(4-pyrimidin-2-ylpiperazin-1-yl)pyridazine) when administered to a subject.

Pharmaceutically acceptable acid addition salts of the present invention, in particular, chloride salts of a compound of the formula I, may result in a reduction or reversal in a subject of one or more of the following: inflammation (e.g. neuroinflammation); activation of signaling pathways involved in inflammation (e.g., neuroinflammation); cell signaling molecule production; activation of glia or glial activation pathways and responses; proinflammatory cytokines or chemokines (e.g., interleukin (IL), in particular IL-1β or tumor necrosis factor (TNF, in particular TNFα); activation of astrocytes or astrocyte activation pathways and responses; activation of microglia or microglial activation pathways and responses; oxidative stress-related responses such as nitric oxide synthase production and nitric oxide accumulation; acute phase proteins; loss of synaptophysin and/or PSD-95; components of the complement cascade; loss or reduction of synaptic function; protein kinase activity (e.g., death associated protein kinase activity); cell damage (e.g., neuronal cell damage); cell death (e.g., neuronal cell death); amyloid βdeposition of amyloid plaques; and behavioral deficits. In aspects of the invention, pharmaceutically acceptable salts of a compound of the formula I result in one or more of the following: suppression of IL-1β, TNFα and S100B up-regulation; decrease in astrocyte (GFAP) and microglia (F4/80) activation; prevention of loss of synaptophysin and/or PSD-95; and attenuation of behavioral deficits

The invention further relates to a method of treating a disease disclosed herein in a subject comprising administering an effective amount of a salt, formulation, dosage form or composition according to the invention. The invention also relates to the use of a salt, formulation, dosage form or composition according to the invention in the preparation of a medicament for treating a disease disclosed herein.

Salts, formulations, dosage forms and compositions of the invention may be administered therapeutically or prophylactically to treat a disease disclosed herein, in particular a neuroinflanunatory disease. Therefore, the invention provides a method for treating a disease disclosed herein, in particular a neuroinflammatory disease, comprising administering a therapeutically effective amount or prophylactically effective amount of an acid addition salt of a compound of the formula I.

Aspects of the invention provide a method of treating a disclosed disease which includes exposing a subject in need of such exposure to a therapeutically effective amount of an acid addition salt of a compound of the formula I, or a prodrug thereof.

In particular aspects of the invention, a method is provided for treating in a subject a disease involving or characterized by inflammation, in particular neuroinflammation, comprising administering to the subject a pharmaceutically acceptable acid addition salt of a compound of the formula I in a therapeutically effective amount that provides beneficial effects, inn pharmaceutically acceptable carrier, excipient, or vehicle.

In a further aspect, the invention provides a method involving administering to a subject a therapeutically effective amount of an acid addition salt of a compound of the formula I, or a formulation, dosage form or composition comprising an acid addition salt of a compound of the Formula I and a pharmaceutically acceptable carrier, excipient, or vehicle which inhibit or reduce neuroinflammation, activation of glia, activation of astrocytes, activation of microglia, proinflammatory cytokines, oxidative stress-related enzymes, acute phase proteins and/or components of the Complement cascade, and/or provide lower risk of QT-related side effects and/or a beneficial pharmacokinetic profile.

The invention also provides a kit comprising one or more acid addition salt of a compound of the formula I, or a formulation, dosage form or composition of the invention. In an aspect, the invention provides a kit for preventing and/or treating a disease disclosed herein, comprising a formulation, dosage form, or composition of the invention, a container, and instructions for use.

Knowledge obtained concerning the acid addition salts of a compound of the formula I may be used to model the tertiary structure of related compounds i.e. analogs and derivatives of compounds of the formula I and salts thereof. In addition, the knowledge of the structure of acid addition salts of a compound of the formula I provides a means of investigating the mechanism of action of these compounds in the body. For example, the ability of compounds to inhibit neuroinflammation, signaling pathways involved in neuroinflammation, proinflammatory cytokines or chemokines (e.g., interleukin (IL) or tumor necrosis factor (TNF), etc., may be predicted by various computer models. The knowledge of the structure of an addition salt of a compound of the formula I, and in particular atomic coordinates and atomic details of a crystalline acid addition salt of a compound of the formula I, may be used to design, evaluate computationally, synthesize and use a compound of the formula I and analogues and derivatives thereof, that prevent or treat any undesirable physical and pharmacological properties of a compound of the formula I. Accordingly, another aspect of the invention is to provide material which is a starting material in the rational design of drugs which mimic the action of acid addition salts of a compound of the formula I. These drugs may be used as therapies that are beneficial in the treatment of diseases disclosed herein.

These and other aspects of the present invention will become evident upon reference to the following detailed description and attached drawings. In addition, reference is made herein to various publications, which are hereby incorporated by reference in their entirety.

### DESCRIPTION OF THE FIGURES

FIG. 1 depicts a production scheme for synthesis of 2-(4-(4-methyl-6-phenylpyridazin-3-yl)piperazin-1-yl)pyrimidine dihydrochloride salt (MW01-9-034WH, also referred to herein as “Minozac”). Reagents and conditions: (a) N₂H₄, EtOH; reflux, (b) CuCl₂, CH₃CN, reflux, (c) POCl₃, CH₃CN, reflux, (d) 1-(2-pyrimidyl)piperazine, water, reflux, (e) HCl, isopropanol.

FIG. 2 shows the in vivo functions of MW01-9-034WH. (A) Compound levels detected in plasma and brain extracts after a single oral gavage administration (2.5 mg/kg) to mice. (B) No histological liver toxicity after oral administration (2.5 mg/kg) to mice once daily for 2 weeks (left); after oral administration of increasing doses up to 100 mg/kg to mice once daily for 3 days (center); and no cardiotoxicity after oral administration (15 mg/kg) to guinea pigs (right) compared to positive control compound, sotalol. (C) Compound suppresses IL-1β, TNFα and S100B up-regulation; decreases astrocyte (GFAP) and microglia (F4/80) activation; prevents loss of synaptophysin and/or PSD-95; and attenuates Y-maze behavioral deficits. C=control; I=Aβ-injured; I+cmpd=Aβ-injured+oral administration (2.5 mg/kg once daily for two weeks beginning three weeks after injury). Data are mean±SEM of n=5-10 mice per group. Significantly different from Aβ-injured: *p<0.05, **p<0.01, ***p<0.001.

FIG. 3 shows in vitro metabolic stability in rat and human liver microsomes. In particular FIG. 3 are bar graphs showing that MW01-9-034WH has improved metabolic stability in rat (A) and human (B) liver microsome incubations compared to minaprine. MW01-9-034WH (5 μM or 25 μM) was incubated with rat or human microsomes for 10 or 30 min. Extracts were prepared, and the amount of compound remaining was analyzed by HPLC using internal standards for recovery. Note that the majority of minaprine is gone by 10 min, but that MW01-9-034WH is stable even after 30 min incubation with rat or human microsomes.

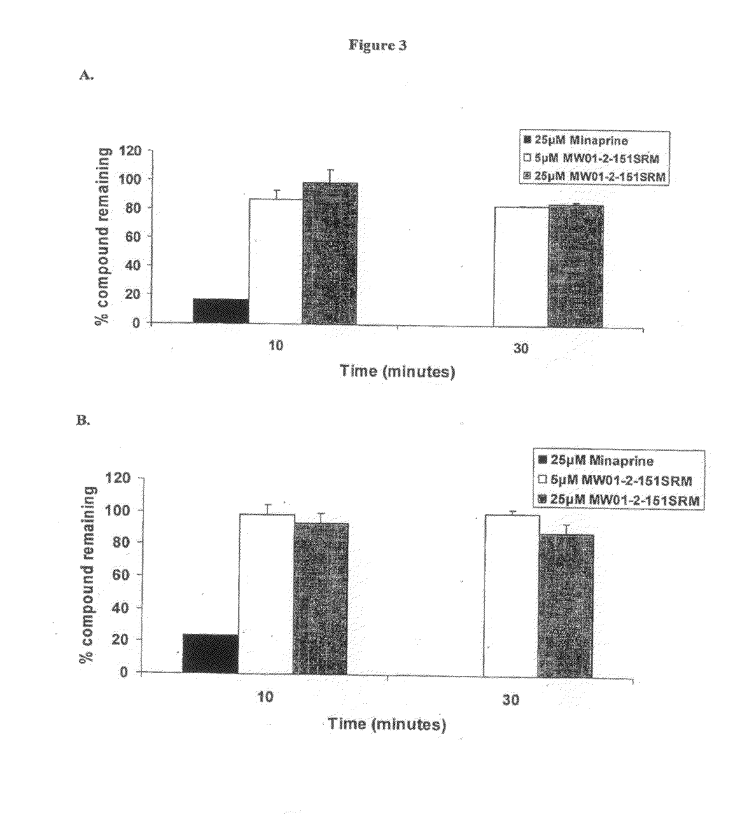

FIG. 4 shows effect of Minozac on systemic proinflammatory cytokine expression.

FIG. 5 shows plasma and brain levels of Minozac in mouse after a single oral dose.

FIG. 6 shows plasma concentration of Minozac after oral dosing with neutralized propylene glycol formulation.

FIG. 7 shows plasma concentration of Minozac after oral dosing with filled gelatin capsule.

### DETAILED DESCRIPTION OF EMBODIMENTS

For convenience, certain terms employed in the specification, examples, and appended claims are collected here.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a,” “an,” and “the” include plural referents unless the content clearly dictates otherwise. Thus, for example, reference to a composition comprising “a compound” includes a mixture of two or more compounds.

As used herein the terms “administering” and “administration” refer to a process by which a therapeutically effective amount of a salt of compound of the formula I or formulation, dosage form, or composition contemplated herein is delivered to a subject for prevention and/or treatment purposes. Formulations, dosage forms, compositions are administered in accordance with good medical practices taking into account the subject's clinical condition, the site and method of administration, dosage, patient age, sex, body weight, and other factors known to physicians.

As used herein, the term “co-administration” or “co-administered” refers to the administration of at least two compounds or agent(s) or therapies to a subject. In some embodiments, the co-administration of two or more agents/therapies is concurrent. In other embodiments, a first agent/therapy is administered prior to a second agent/therapy. In this aspect, each component may in administered separately, but sufficiently close in time to provide the desired effect, in particular a beneficial, additive, or synergistic effect. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

The term “treating” refers to reversing, alleviating, or inhibiting the progress of a disease, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a compound or composition of the present invention to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above. The purpose of prevention and intervention is to combat the disease, condition, or disorder and includes the administration of the active compounds to prevent or delay the onset of the symptoms or complications, or alleviating the symptoms or complications, or eliminating the disease, condition, or disorder.

The terms “subject”, “individual”, or “patient” are used interchangeably herein and refer to an animal preferably a warm-blooded animal such as a mammal. Mammal includes without limitation any members of the Mammalia. A mammal, as a subject or patient in the present disclosure, can be from the family of Primates, Carnivora, Proboscidea, Perissodactyla, Artiodactyla, Rodentia, and Lagomorpha. Among other specific embodiments a mammal of the present invention can be Canis familiaris (dog), Felis catus (cat), Elephas maximus (elephant), Equus caballus (horse), Sus domesticus (pig), Camelus dromedarious (camel), Cervus axis (deer), Giraffa camelopardalis (giraffe), Bos Laurus (cattle/cows), Capra hircus (goat), Ovis aries (sheep), Mus musculus (mouse), Lepus brachyurus (rabbit), Mesocricetus auratus (hamster), Cavia porcellus (guinea pig), Meriones unguiculatus (gerbil), or Homo sapiens (human). In a particular embodiment, the mammal is a human. In other embodiments, animals can be treated; the animals can be vertebrates, including both birds and mammals. In aspects of the invention, the terms include domestic animals bred for food or as pets, including equines, bovines, sheep; poultry, fish, porcines, canines, felines, and zoo animals, goats, apes (e.g. gorilla or chimpanzee), and rodents such as rats and mice.

Typical subjects for treatment include persons afflicted with or suspected of having or being pre-disposed to a disease disclosed herein, or persons susceptible to, suffering from or that have suffered a disease disclosed herein. A subject may or may not have a genetic predisposition for a disease disclosed herein. In the context of certain aspects of the invention, the term “subject” generally refers to an individual who will receive or who has received treatment (e.g., administration of an acid addition salt of a compound of the formula I, and optionally one or more other agents) for a condition characterized by inflammation, the dysregulation of protein kinase activity, and/or dysregulation of apoptotic processes. In certain aspects of the invention, the subject is a healthy subject.

In particular aspects, a subject shows signs of cognitive deficits or Alzheimer's disease neuropathology. In embodiments of the invention the subjects are susceptible to, or suffer from Alzheimer's disease. In embodiments of the invention, a subject is receiving a therapeutic or treatment that prolongs QT interval.

As utilized herein, the term “healthy subject” means a subject, in particular a mammal, having no diagnosed disease, disorder, infirmity, or ailment, more particularly a disease, disorder, infirmity or ailment known to impair or otherwise diminish memory.

The term “diagnosed,” as used herein, refers to the recognition of a disease by its signs and symptoms (e.g., resistance to conventional therapies), or genetic analysis, pathological analysis, histological analysis, and the like.

A “halide salt” is a chloride, fluoride, bromide, iodide salt, preferably, a chloride or bromide salt. The counter-cation of the salt can be an alkali metal (e.g. Li, Na, or K), or preferably, hydrogen.

“Therapeutically effective amount” relates to the amount or dose of an acid addition salt of a compound of the formula I or formulation, dosage form or composition comprising the same, that will lead to one or more desired effects, in particular, one or more beneficial effects. A therapeutically effective amount of a substance can vary according to factors such as the disease state, age, sex, and weight of the subject, and the ability of the substance to elicit a desired response in the subject. A dosage regimen may be adjusted to provide the optimum therapeutic response or pharmacokinetic profile. For example, several divided doses may be administered daily or the dose may be proportionally reduced as indicated by the exigencies of the therapeutic situation.

The term “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

A “beneficial effect” refers to an effect of a salt, formulation, dosage form, or composition according to the invention including favorable pharmacological and/or therapeutic effects, improved biological activity, and improved physical properties. In aspects of the invention, the beneficial effects include without limitation prevention, reduction, reversal, or inhibition of one or more of the following: inflammation (e.g. neuroinflammation), activation of signaling pathways involved in inflammation (e.g., neuroinflammation), cell signaling molecule production, activation of glia or glial activation pathways and responses, proinflammatory cytokines or chemokines (e.g., interleukin (IL), in particular IL-1β or tumor necrosis factor (TNF, in particular TNFα), activation of astrocytes or astrocyte activation pathways and responses; activation of micoglia or microglial activation pathways and responses; oxidative stress-related responses such as nitric oxide synthase production and nitric oxide accumulation, acute phase proteins, loss of synaptophysin and/or PSD-95, components of the complement cascade, protein kinase activity (e.g., death associated protein kinase activity), amyloid β deposition of amyloid plaques, loss or reduction of synaptic function, cell damage (e.g., neuronal cell damage), behavioral deficits, and/or cell death (e.g., neuronal cell death). In some aspects, a beneficial effect is a favourable characteristic, including without limitation enhanced stability, enhanced solubility, increased rate of dissolution, increased terminal stability, enhanced bioavailability, a longer half life, reduced risk of side effects, and/or enhanced uptake and transport across the blood brain barrier or enhanced brain penetrance. In particular aspects, the beneficial effect is enhanced stability, enhanced solubility, increased rate of dissolution, increased terminal stability, and/or enhanced bioavailability.

In an embodiment, where the disease is Alzheimer's disease, beneficial effects of a salt, formulation, dosage form, composition or treatment of the invention can manifest as one, two, three, four, five, six, seven, eight, or all of the following, in particular five or more, more particularly seven or more of the following:
a) A reduction in protein kinase activity (e.g. DAPK), in particular at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, or 99% decrease in protein kinase activity.
b) A reduction in glial activation response, in particular, at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, or 99% reduction in glial activation response.
c) A reduction in glial activity in the brain, relative to the levels determined in the absence of a salt, formulation, dosage form, composition or treatment according to the invention or relative to a parent or lead compound in subjects with symptoms of Alzheimer's disease. In particular, the compounds induce at least about a 2%, 5%, 10%, 15%, 20%, 30%, 40%, 50%, 60%, 70%, 80%, or 90% decrease in glial activity.
d) A reduction in astrocyte activation response, in particular, at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, or 99% reduction in astrocyte activation.
e) A reduction in astrocyte activity in the brain, relative to the levels determined in the absence of a salt, formulation, dosage form, composition or treatment according to the invention or relative to a parent or lead compound in subjects with symptoms of Alzheimer's disease. In particular, the compounds induce at least about a 2%, 5%, 10%, 15%, 20%, 30%, 40%, 50%, 60%, 70%, 80%, or 90% decrease in astrocyte activity.
f) A reduction in microglial activation, in particular, at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, or 99% reduction in microglial activation.
g) A reduction in microglial activation response, in particular, at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, or 99% reduction in microglial activation response.
h) A reduction in loss of synaptophysin and/or PSD-95, in particular at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, or 99% reduction in loss of synaptophysin and/or PSD-95.

i) A reduction in oxidative stress-related responses (e.g., nitric oxide synthase production and/or nitric oxide accumulation), in particular at least about a 0.05%; 0.1%, 0.5%, 1%, 2%, 5%, 10%, 15%, 20%, 30%, 33%, 35%, 40%, 45%, 50%, 60%, 70%, 80%, 90%, 95%, or 99% reduction in oxidative stress-related responses such as nitric oxide synthase production and nitric oxide accumulation.
j) A reduction in cellular apoptosis and/or death associated protein kinase activity, in particular a 0.05%, 0.1%, 0.5%, 1%, 2%, 5%, 10%, 15%, 20%, 30%, 33%, 35%, 40%, 45%, 50%, 60%, 70%, 80%, 90%, 95%, or 99% reduction in cellular apoptosis and/or death associated protein kinase activity.
k) A reduction in proinflammatory cytokine responses in particular a 0.05%, 0.1%, 0.5%, 1%, 2%, 5%, 10%, 15%, 20%, 30%, 33%, 35%, 40%, 45%, 50%, 60%, 70%, 80%, 90%, 95%, or 99% reduction in proinflammatory cytokine responses.
l) A reduction in interleukin-1β and/or tumor necrosis factor α production in particular a 0.05%, 0.1%, 0.5%, 1%, 2%, 5%, 10%, 15%, 20%, 30%; 33%, 35%, 40%, 45%, 50%, 60%, 70%, 80%, 90%, 95%, or 99% reduction in interleukin-1β and/or tumor necrosis factor α production.
m) A slowing of the rate of disease progression in a subject with Alzheimer's disease.
n) Increase in survival in a subject with symptoms of Alzheimer's disease.

In particular aspects of the invention beneficial effects of salts, formulations, dosage forms, compositions or treatments of the invention can manifest as (a) and (b); (a), (b) and (c); (a) through (d); (a) through (e); (a) through (f); (a) through (g); (a) through (h); (a) through (i), (a) through (j), (a) through (k), (a) through (l), (a) through (m), or (a) through (n).

The term “beneficial pharmacokinetic profile” refers to amounts or doses of a compound of the formula I that provide levels of the compound in plasma and/or brain or a required dose resulting in therapeutic effects in the prevention, treatment, or control of symptoms of a disease disclosed herein, in particular a neuroinflammatory disease, more particularly Alzheimer's disease. A beneficial pharmacokinetic profile may be a sustained pharmacokinetic profile. The term “sustained pharmacokinetic profile” as used herein refers to a length of time efficacious levels of a biologically active compound of the formula I is in its environment of use. A sustained pharmacokinetic profile can be such that a single or twice daily administration adequately prevents, treats, or controls symptoms of a disease disclosed herein. A beneficial pharmacokinetic profile may provide therapeutically effective amounts of the compound of the formula I in the plasma and/or brain for about 12 to about 48 hours, 12 hours to about 36 hours, or 12 hours to about 24 hours.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. Examples of carriers etc. include but are not limited to saline, buffered saline, dextrose, water, glycerol, ethanol, and combinations thereof. The use of such media and agents for an active substance is well known in the art.

An acid addition salt of a compound of the Formula I can contain one or more asymmetric centers and may give rise to enantiomers, diasteriomers, and other stereoisomeric forms which may be defined in terms of absolute stereochemistry as (R)- or (S)-. Thus, salts of compounds of the Formula I include all possible diasteriomers and enantiomers as well as their racemic and optically pure forms. Optically active (R)-, and (S)-isomers may be prepared using chiral synthons or chiral reagents, or resolved using conventional techniques. When a salt of a compound of the Formula I contains centers of geometric asymmetry, and unless specified otherwise, it is intended that the compounds include both E and A geometric isomers. All tautomeric forms are also included within the scope of a salt of a compound of the Formula I.

A salt of a compound of the Formula I may be pure or substantially pure. As used herein, the term “pure” in general means better than 90%, 92%, 93%, 94%, 95%, 96%, 97%, 98% or 99% pure, and “substantially pure” means a compound synthesized such that the compound, as made or as available for consideration into a composition or therapeutic dosage described herein, has only those impurities that can not readily nor reasonably be removed by conventional purification processes.

“Optional” or “optionally” means that the subsequently described event or circumstance may but need not occur, and that the description includes instances where the event or circumstance occurs and instances in which it does not occur. For example, “alkyl group optionally substituted with a halo group” means that the halo may but need not be present, and the description includes situations where the alkyl group is substituted with a halo group and situations where the alkyl group is not substituted with the halo group.

A salt of a compound of the Formula I includes derivatives. As used herein the term “derivative” refers to a chemically modified compound wherein the chemical modification takes place either at a functional group or ring of the compound. Non-limiting examples of derivatives may include N-acetyl, N-methyl, N-hydroxy groups at any of the available nitrogens in the compound. A “derivative” also includes a functional derivative of a salt of a compound of the formula I. A “functional derivative” refers to a compound that possesses a biological activity (either functional or structural) that is substantially similar to the biological activity of a salt of a compound of the formula I. The term “functional derivative” is intended to include “variants” “analogs” or “chemical derivatives” of a salt of a compound of the formula I. The term “variant” is meant to refer to a molecule substantially similar in structure and function to a salt of a compound of the formula I or a part thereof. A molecule is “substantially similar” to another compound if both molecules have substantially similar structures or if both molecules possess similar biological activity. The term “analog” refers to a molecule substantially similar in function to a salt according to the invention. The term “chemical derivative” describes a molecule that contains additional chemical moieties which are not normally a part of the base molecule.

In aspects of the invention, an acid addition salt of a compound of the formula I is a pharmaceutically functional derivative. A “pharmaceutically functional derivative” includes any pharmaceutically acceptable derivative of a salt of a compound of the formula I, for example, an ester or an amide, which upon administration to a subject is capable of providing (directly or indirectly) a salt of a compound of the formula I, or an active metabolite or residue thereof. Such derivatives are recognizable to those skilled in the art, without undue experimentation (see for example Burger's Medicinal Chemistry and Drug Discovery, 5.sup.th Edition, Vol 1: Principles and Practice, which has illustrative pharmaceutically functional derivatives).

The term “alkyl”, either alone or within other terms such as “thioalkyl” and “arylalkyl”, means a monovalent, saturated hydrocarbon radical which may be a straight chain (i.e. linear) or a branched chain. An alkyl radical for use in the present invention generally comprises from about 1 to 20 carbon atoms, particularly from about 1 to 10, 1 to 8 or 1 to 7, more particularly about 1 to 6 carbon atoms, or 3 to 6 carbon atoms. Illustrative alkyl radicals include methyl, ethyl, n-propyl, n-butyl, n-pentyl, n-hexyl, isopropyl, isobutyl, isopentyl, amyl, sec-butyl, tert-butyl, tert-pentyl, n-heptyl, n-octyl, n-nonyl, n-decyl, undecyl, n-dodecyl, n-tetradecyl, pentadecyl, n-hexadecyl, heptadecyl, n-octadecyl, nonadecyl, eicosyl, dosyl, n-tetracosyl, and the like, along with branched variations thereof. In certain aspects of the invention an alkyl radical is a C₁-C₆ lower alkyl comprising or selected from the group consisting of methyl, ethyl, n-propyl, n-butyl, n-pentyl, n-hexyl, isopropyl, isobutyl, isopentyl, amyl, tributyl, sec-butyl; tert-butyl, tert-pentyl, and n-hexyl.

An alkyl radical may be optionally substituted with substituents as defined herein at positions that do not significantly interfere with the preparation of compounds of the formula I and do not significantly reduce the efficacy of the compounds. In certain aspects of the invention, an alkyl radical is substituted, in particular with one to five substituents; including halo, lower alkoxy, lower aliphatic, a substituted lower aliphatic, hydroxy, cyano, nitro, thio, amino, keto, aldehyde, ester, amide, substituted amino, carboxyl, sulfonyl, sulfonyl, sulfenyl, sulfate, sulfoxide, substituted carboxyl, halogenated lower alkyl (e.g. CF₃), halogenated lower alkoxy, hydroxycarbonyl, lower alkoxycarbonyl, lower alkylcarbonyloxy, lower alkylcarbonylamino, cycloaliphatic, substituted cycloaliphatic, or aryl (e.g., phenylmethyl (i.e. benzyl)), heteroaryl (e.g., pyridyl), and heterocyclic (e.g., piperidinyl, morpholinyl). Substituents on an alkyl group may themselves be substituted.

In respect to certain aspects of the invention, the term “substituted aliphatic” refers to an alkyl or an alkane possessing less than 10 carbons where at least one of the aliphatic hydrogen atoms has been replaced by a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic, etc.). Examples of such groups include, but are not limited to, 1-chloroethyl and the like.

As used herein the term “alkenyl” refers to an unsaturated, acyclic branched or straight-chain hydrocarbon radical comprising at least one double bond. An alkenyl radical may contain from about 2 to 24 or 2 to 10 carbon atoms; in particular from about 3 to 8 carbon atoms and more particularly about 3 to 6 or 2 to 6 carbon atoms. Suitable alkenyl radicals include without limitation ethenyl, propenyl (e.g., prop-1-en-1-yl, prop-1-en-2-yl, prop-2-en-1-yl (allyl), prop-2-en-2-yl), buten-1-yl, but-1-en-2-yl, 2-methyl-prop-1-en-1-yl, but-2-en-1-yl, but-2-en-2-yl, buta-1,3-dien-1-yl, buta-1,3-dien-2-yl, hexen-1-yl, 3-hydroxyhexen-1-yl, hepten-1-yl, and octen-1-yl, and the like. An alkenyl radical may be optionally substituted similar to alkyl.

A used herein, the term “cycloalkenyl” refers to radicals comprising about 4 to 16, 2 to 15, 2 to 10, 2 to 8, 4 to 10, 3 to 8, 3 to 7, 3 to 6, or 4 to 6 carbon atoms, one or more carbon-carbon double bonds, and one, two, three, or four rings wherein such rings may be attached in a pendant manner or may be fused. In certain aspects of the invention the cycloalkenyl radicals are “lower cycloalkenyl” radicals having three to seven carbon atoms. Examples of cycloalkenyl radicals include without limitation cyclobutenyl, cyclopentenyl, cyclohexenyl and cycloheptenyl. A cycloalkenyl radical may be optionally substituted with groups as disclosed herein, in particular 1, 2, or 3 substituents which may be the same or different.

As used herein, the term “alkynyl” refers to an unsaturated, branched or straight-chain hydrocarbon radical comprising one or more triple bonds. An alkynyl radical may contain about 1 to 20, 1 to 15, or 2 to 10 carbon atoms, particularly about 3 to 8 carbon atoms and more particularly about 3 to 6 carbon atoms. Suitable alkynyl radicals include without limitation ethynyl, such as prop-1-yn-1-yl, and prop-2-yn-1-yl, butynyls such as but-1-yn-1-yl, but-1-yn-3-yl, and but-3-yn-1-yl, pentynyls such as pentyn-1-yl, pentyn-2-yl, 4-methoxypentyn-2-yl, and 3-methylbutyn-1-yl, hexynyls such as hexyn-1-yl, hexyn-2-yl, hexyn-3-yl, and 3,3-dimethylbutyn-1-yl radicals and the like. In aspects of the invention, alkenyl groups include ethenyl (—CH═CH₂), n-propenyl (—CH₂CH═CH₂), iso-propenyl (—C(CH₃)═CH₂), and the like. An alkenyl may be optionally substituted similar to alkyl. The term “cycloalkynyl” refers to cyclic alkynyl groups.

As used herein the term “alkylene” refers to a linear or branched radical having from about 1 to 10, 1 to 8, 1 to 6, or 2 to 6 carbon atoms and having attachment points for two or more covalent bonds. Examples of such radicals are methylene, ethylene, propylene, butylene, pentylene, hexylene, ethylidene, methylethylene, and isopropylidene. When an alkenylene radical is present as a substituent on another radical it is typically considered to be a single substituent rather than a radical formed by two substituent.

As used herein the term “alkenylene” refers to a linear or branched radical having from about 2 to 10, 2 to 8 or 2 to 6 carbon atoms, at least one double bond, and having attachment points for two or more covalent bonds. Examples of alkenylene radicals include 1,1-vinylidene (—CH2=C—), 1,2-vinylidene (—CH═CH—), and 1,4-butadienyl (—CH═CH—CH═CH—).

As used herein the term “halo” refers to a halogen such as fluorine, chlorine, bromine or iodine atoms.

As used herein the term “hydroxyl” or “hydroxy” refers to an —OH group.

As used herein the term “cyano” refers to a carbon radical having three of four covalent bonds shared by a nitrogen atom, in particular —C≡N. A cyano group may be substituted with substituents described herein.

As used herein the term “alkoxy” refers to a linear or branched oxy-containing radical having an alkyl portion of one to about ten carbon atoms, such as a methoxy radical, which may be substituted. In aspects of the invention an alkoxy radical may comprise about 1-10, 1-8, 1-6 or 1-3 carbon atoms. In embodiments of the invention, an alkoxy radical comprises about 1-6 carbon atoms and includes a C₁-C₆ alkyl-O-radical wherein C₁-C₆ alkyl has the meaning set out herein. Examples of alkoxy radicals include without limitation methoxy, ethoxy, propoxy, butoxy, isopropoxy and tert-butoxy alkyls. An “alkoxy” radical may optionally be substituted with one or more substitutents disclosed herein including alkyl atoms to provide “alkylalkoxy” radicals; halo atoms, such as fluoro, chloro or bromo, to provide “haloalkoxy” radicals (e.g. fluoromethoxy, chloromethoxy, trifluoromethoxy, difluoromethoxy, trifluorethoxy, fluoroethoxy, tetrafluoroethoxy, pentafluoroethoxy, and fluoropropox) and “haloalkoxyalkyl” radicals (e.g. fluoromethoxymethyl, chloromethoxyethyl, trifluoromethoxymethyl, difluoromethoxyethyl, and trifluoroethoxymethyl).

As used herein the term “cycloalkoxy” refers to cycloalkyl radicals (in particular, cycloalkyl radicals having 3 to 15, 3 to 8 or 3 to 6 carbon atoms) attached to an oxy radical. Examples of cycloalkoxy radicals include cyclohexoxy and cyclopentoxy. A cycloalkoxy radical may be optionally substituted with groups as disclosed herein.

As used herein the term “alkenyloxy” refers to linear or branched oxy-containing radicals having an alkenyl portion of about 2 to 10 carbon atoms, such as an ethenyloxy or propenyloxy radical. An alkenyloxy radical may be a “lower alkenyloxy” radical having about 2 to 6 carbon atoms. Examples of alkenyloxy radicals include without limitation ethenyloxy, propenyloxy, butenyloxy, and isopropenyloxy alkyls. An “alkenyloxy” radical may be substituted with one or more substitutents disclosed herein including halo atoms, such as fluoro, chloro or bromo, to provide “haloalkenyloxy” radicals (e.g. trifluoroethenyloxy, fluoroethenyloxy, difluoroethenyhioxy, and fluoropropenyloxy).

As used herein, the term “cycloalkyl” refers to radicals having from about 3 to 16 or 3 to 15 carbon atoms and containing one, two, three, or four rings wherein such rings may be attached in a pendant manner or may be fused. In aspects of the invention, “cycloalkyl” refers to an optionally substituted, saturated hydrocarbon ring system containing 1 to 2 rings and 3 to 7 carbons per ring which may be further fused with an unsaturated C₃-C₇ carbocylic ring. Examples of cycloalkyl groups include single ring structures such as cyclopropyl, cyclobutyl, cyclopentyl, cyclohexyl, cycloheptyl, cyclooctyl, cyclononyl, cyclodecyl, cyclododecyl, and the like, or multiple ring structures such as adamantanyl, and the like. In certain aspects of the invention the cycloalkyl radicals are “lower cycloalkyl” radicals having from about 3 to 10, 3 to 8, 3 to 6, or 3 to 4 carbon atoms, in particular cyclopropyl, cyclobutyl, cyclopentyl, cyclohexyl and cycloheptyl. The term “cycloalkyl” also embraces radicals where cycloalkyl radicals are fused with aryl radicals or heterocyclyl radicals. A cycloalkyl radical may be optionally substituted with groups as disclosed herein.

In aspects of the invention, “substituted cycloalkyl” refers to cycloalkyl groups having from 1 to 5 (in particular 1 to 3) substituents including without limitation alkyl, alkenyl, alkoxy, cycloalkyl, substituted cycloalkyl, acyl, acylamino, acyloxy, amino, aminoacyl, aminoacyloxy, oxyacylamino, cyano, halogen, hydroxyl, carboxyl, carboxylalkyl, keto, thioketo, thiol, thioalkoxy, aryl, aryloxy, heteroaryl, heteroaryloxy, hydroxyamino, alkoxyamino, and nitro.

As used herein in respect to certain aspects of the invention, the term “cycloaliphatic” refers to a cycloalkane possessing less than 8 carbons or a fused ring system consisting of no more than three fused cycloaliphatic rings. Examples of such groups include, but are not limited to, decalin and the like.

As used herein in respect to certain aspects of the invention, the term “substituted cycloaliphatic” refers to a cycloalkane possessing less than 8 carbons or a fused ring system consisting of no more than three fused rings, and where at least one of the aliphatic hydrogen atoms has been replaced by a halogen, a nitro, a thio, an amino, a hydroxy, a ketone, an aldehyde, an ester, an amide, a lower aliphatic, a substituted lower aliphatic, or a ring (aryl, substituted aryl, cycloaliphatic, or substituted cycloaliphatic). Examples of such groups include, but are not limited to, 1-chlorodecalyl and the like

As used herein, the term “aryl”, alone or in combination, refers to a carbocyclic aromatic system containing one, two or three rings wherein such rings may be attached together in a pendant manner or may be fused. In aspects of the invention an aryl radical comprises 4 to 24 carbon atoms, in particular 4 to 10, 4 to 8, or 4 to 6 carbon atoms. Illustrative “aryl” radicals include without limitation aromatic radicals such as phenyl, benzyl, naphthyl, indenyl, benzocyclooctenyl, benzocycloheptenyl, pentalenyl, azulenyl, tetrahydronaphthyl, indanyl, biphenyl, biphenyl, acephthylenyl, fluorenyl, phenalenyl, phenanthrenyl, and anthracenyl, preferably phenyl.

An aryl radical may be optionally substituted with groups as disclosed herein, in particular hydroxyl, alkyl (i.e., arylalkyl), carbonyl, carboxyl, thiol (i.e., thiolaryl), amino, and/or halo, in particular a substituted aryl includes without limitation arylamine and arylalkyamine.

As used herein in respect to certain aspects of the invention, the term “substituted aryl” refers to an aromatic ring, or fused aromatic ring system consisting of no more than three fused rings at least one of which is aromatic, and where at least one of the hydrogen atoms on a ring carbon has been replaced by a halogen, an amino, a hydroxy, a nitro, a thio, an alkyl, a ketone, an aldehyde, an ester, an amide, a lower aliphatic, a substituted lower aliphatic, or a ring (aryl, substituted aryl, cycloaliphatic, or substituted cycloaliphatic). Examples of substituted aryl groups include, but are not limited to, hydroxyphenyl, chlorophenyl and the like.

As used herein, the term “aryloxy” refers to aryl radicals, as defined above, attached to an oxygen atom. Exemplary aryloxy groups include napthyloxy, quinolyloxy, isoquinolizinyloxy, and the like.

As used herein the term “arylalkoxy,” refers to an aryl group attached to an alkoxy group. Representative examples of arylalkoxy include, but are not limited to, 2-phenylethoxy, 3-naphth-2-ylpropoxy, and 5-phenylpentyloxy.

As used herein, the term “aroyl” refers to aryl radicals, as defined above, attached to a carbonyl radical as defined herein, including without limitation benzoyl and toluoyl. An aroyl radical may be optionally substituted with groups as disclosed herein.

As used herein the term “heteroaryl” refers to fully unsaturated heteroatom-containing ring-shaped aromatic radicals having at least one heteroatom selected from carbon, nitrogen, sulfur and oxygen. A heteroaryl radical may contain one, two or three rings and the rings may be attached in a pendant manner or may be fused. In aspects of the invention the term refers to fully unsaturated heteroatom-containing ring-shaped aromatic radicals having from 3 to 15, 3 to 10, 3 to 8, 5 to 15, 5 to 10, or 5 to 8 ring members selected from carbon, nitrogen, sulfur and oxygen, wherein at least one ring atom is a heteroatom. Examples of “heteroaryl” radicals, include without limitation, an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, tetrazolyl and the like; an unsaturated condensed heterocyclic group containing 1 to 5 nitrogen atoms, in particular, indolyl, isoindolyl, indolizinyl, benzimidazolyl, quinolyl, indazolyl, quinazolinyl, pteridinyl, quinolizidinyl, phthalazinyl, naphthyridinyl, quinoxalinyl, cinnolinyl, phenanthridinyl, acridinyl, phenanthrolinyl, phenazinyl, carbazolyl, purinyl, benzimidazolyl, quinolinyl, isoquinolinyl, benzotriazolyl, tetrazolopyridazinyl and the like; an unsaturated 3 to 6-membered heteromonocyclic group containing an oxygen atom, in particular, 2-furyl, 3-furyl, pyranyl, and the like; an unsaturated 5 to 6-membered heteromonocyclic group containing a sulfur atom, in particular, thienyl, 2-thienyl, 3-thienyl, and the like; unsaturated 5 to 6-membered heteromonocyclic group containing 1 to 2 oxygen atoms and 1 to 3 nitrogen atoms, in particular, furazanyl, benzofurazanyl, oxazolyl, isoxazolyl, and oxadiazolyl; an unsaturated condensed heterocyclic group containing 1 to 2 oxygen atoms and 1 to 3 nitrogen atoms, in particular benzoxazolyl, benzoxadiazolyl and the like; an unsaturated 5 to 6-membered heteromonocyclic group containing 1 to 2 sulfur atoms and 1 to 3 nitrogen atoms, for example, thiazolyl, isothiazolyl, thiadiazolyl and the like; an unsaturated condensed heterocyclic group containing 1 to 2 sulfur atoms and 1 to 3 nitrogen atoms such as benzothiazolyl, benzothiadiazolyl and the like. The term also includes radicals where heterocyclic radicals are fused with aryl radicals, in particular bicyclic radicals such as benzofuranyl, benzothiophenyl, phthalazinyl, chromenyl, xanthenyl, and the like. A heteroaryl radical may be optionally substituted with groups as disclosed herein, for example with an alkyl, amino, halogen, etc., in particular a heteroarylamino.

In aspects of the invention, the term refers to an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, tetrazolyl and the like.

A heteroaryl radical may be optionally substituted with groups as disclosed herein, for example with an alkyl, amino, halogen, etc., in particular a heteroarylamine.

The term “heterocyclic” refers to saturated and partially saturated heteroatom-containing ring-shaped radicals having at least one heteroatom selected from carbon, nitrogen, sulfur and oxygen. A heterocylic radical may contain one, two or three rings wherein such rings may be attached in a pendant manner or may be fused. In an aspect, the term refers to a saturated and partially saturated heteroatom-containing ring-shaped radicals having from about 3 to 15, 3 to 10, 5 to 15, 5 to 10, or 3 to 8 ring members selected from carbon, nitrogen, sulfur and oxygen, wherein at least one ring atom is a heteroatom. Exemplary saturated heterocyclic radicals include without limitation a saturated 3 to 6-membered heteromonocyclic group containing 1 to 4 nitrogen atoms [e.g. pyrrolidinyl, imidazolidinyl, and piperazinyl]; a saturated 3 to 6-membered heteromonocyclic group containing 1 to 2 oxygen atoms and 1 to 3 nitrogen atoms [e.g. morpholinyl; sydnonyl]; and, a saturated 3 to 6-membered heteromonocyclic group containing 1 to 2 sulfur atoms and 1 to 3 nitrogen atoms [e.g., thiazolidinyl] etc. Examples of partially saturated heterocyclyl radicals include without limitation dihydrothiophene, dihydropyranyl, dihydrofuranyl and dihydrothiazolyl. Illustrative heterocyclic radicals include without limitation aziridinyl, azetidinyl, 2-pyrrolinyl, 3-pyrrolinyl, pyrrolidinyl, azepinyl, 1,3-dioxolanyl, 2H-pyranyl, 4H-pyranyl, piperidinyl, 1,4-dioxanyl, morpholinyl, pyrazolinyl, 1,4-dithianyl, thiomorpholiny 1,1,2,3,6-tetrahydropyridinyl, oxiranyl, oxetanyl, tetrahydrofuranyl, tetrahydropyranyl, tetrahydropyridinyl, tetrahydrothiopyranyl, thioxanyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3H-indolyl, quinuclidinyl, quinolizinyl, and the like.

As used herein in respect to certain aspects of the invention, the term “heterocyclic” refers to a cycloalkane and/or an aryl ring system, possessing less than 8 carbons, or a fused ring system consisting of no more than three fused rings, where at least one of the ring carbon atoms is replaced by oxygen, nitrogen or sulfur. Examples of such groups include, but are not limited to, morpholino and the like.

As used herein in respect to certain aspects of the invention, the term “substituted heterocyclic” refers to a cycloalkane and/or an aryl ring system, possessing less than 8 carbons, or a fused ring system consisting of no more than three fused rings, where at least one of the ring carbon atoms is replaced by oxygen, nitrogen or sulfur, and where at least one of the aliphatic hydrogen atoms has been replaced by a halogen, hydroxy, a thio, nitro, an amino, a ketone, an aldehyde, an ester, an amide, a lower aliphatic, a substituted lower aliphatic, or a ring (aryl, substituted aryl, cycloaliphatic, or substituted cycloaliphatic). Examples of such groups include, but are not limited to 2-chloropyranyl.

The foregoing heteroaryl and heterocyclic groups may be C-attached or N-attached (where such is possible).

As used herein the term “sulfonyl”, used alone or linked to other terms such as alkylsulfonyl or arylsulfonyl, refers to the divalent radicals —SO₂⁻. In aspects of the invention, the sulfonyl group may be attached to a substituted or unsubstituted hydroxyl, alkyl group, ether group, alkenyl group, allynyl group, aryl group, cycloalkyl group, cycloalkenyl group, cycloalkynyl group, heterocyclic group, carbohydrate, peptide, or peptide derivative.

The term “sulfinyl”, used alone or linked to other terms such as alkylsulfinyl (i.e. —S(O)-alkyl) or arylsulfinyl; refers to the divalent radicals —S(O)—.

As used herein the term “amino”, alone or in combination, refers to a radical where a nitrogen atom (N) is bonded to three substituents being any combination of hydrogen, hydroxyl, alkyl, cycloalkyl, alkenyl, alkynyl, aryl, silyl, heterocyclic, or heteroaryl with the general chemical formula —NR¹R² where R¹ and R² can be any combination of hydrogen, hydroxyl, alkyl, cycloalkyl, alkoxy, alkenyl, alkynyl, aryl, carbonyl carboxyl, amino, silyl, heteroaryl, or heterocyclic which may or may not be substituted. Optionally one substituent on the nitrogen atom may be a hydroxyl group (—OH) to provide an amine known as a hydroxylamine. Illustrative examples of amino groups are amino (—NH₂), alkylamino, acylamino, cycloamino, acycloalkylamino, arylamino, arylalkylamino, and lower alkylsilylamino, in particular methylamino, ethylamino, dimethylamino, 2-propylamino, butylamino, isobutylamino, cyclopropylamino, benzylamino, allylamino, hydroxylamino, cyclohexylamino, piperidinyl, hydrazinyl, benzylamino, diphenylmethylamino, tritylamino, trimethylsilylamino, and dimethyl-tert.-butylsilylamino, which may or may not be substituted.

The term “sulfenyl” used alone or linked to other terms such as alkylsulfenyl, refers to the radical —SR³ wherein R³ is not hydrogen. In aspects of the invention R³ is substituted or unsubstituted alkyl, cycloalkyl, alkenyl, alkynyl, aryl, silyl, silylalkyl, heterocyclic, heteroaryl, carbonyl, carbamoyl, alkoxy, or carboxyl.

As used herein the term “thiol” means —SH. A thiol may be substituted with a substituent disclosed herein, in particular alkyl (thioalkyl), aryl (thioaryl), alkoxy (thioalkoxy) or carboxyl.

As used herein, the term “thioalkyl”, alone or in combination, refers to a chemical functional group where a sulfur atom (S) is bonded to an alkyl, which may be substituted. Examples of thioalkyl groups are thiomethyl, thioethyl, and thiopropyl. A thioalkyl may be substituted with a substituted or unsubstituted carboxyl, aryl, heterocylic, carbonyl, or heterocyclic.

As used herein the term “thioaryl”, alone or in combination, refers to a chemical functional group where a sulfur atom (S) is bonded to an aryl group with the general chemical formula —SR⁴ where R⁴ is aryl which may be substituted. Illustrative examples of thioaryl groups and substituted thioaryl groups are thiophenyl, chlorothiophenyl, para-chlorothiophenyl, thiobenzyl, 4-methoxy-thiophenyl, 4-nitro-thiophenyl, and para nitrothiobenzyl.

As used herein the term “thioalkoxy”, alone or in combination, refers to a chemical functional group where a sulfur atom (S) is bonded to an alkoxy group with the general chemical formula SR⁵ where R⁵ is an alkoxy group which may be substituted. A “thioalkoxy group” may have 1-6 carbon atoms i.e. a —S—(O)—C₁-C₆ alkyl group wherein C₁-C₆ alkyl have the meaning as defined above. Illustrative examples of a straight or branched thioalkoxy group or radical having from 1 to 6 carbon atoms, also known as a C₁-C₆ thioalkoxy, include thiomethoxy and thioethoxy.

A thiol may be substituted with a substituted or unsubstituted heteroaryl or heterocyclic, in particular a substituted or unsubstituted saturated 3 to 6-membered heteromonocylic group containing 1 to 4 nitrogen atoms [e.g. pyrrolidinyl, imidazolidinyl, piperidinyl, and piperazinyl] or a saturated 3 to 6-membered heteromonocyclic group containing 1 to 2 oxygen atoms and 1 to 3 nitrogen atoms [e.g. morpholinyl; sydnonyl], especially a substituted morpholinyl or piperidinyl.

As used herein, the term “carbonyl” refers to a carbon radical having two of the four covalent bonds shared with an oxygen atom.

As used herein, the term “carboxyl”, alone or in combination, refers to —C(O)OR⁶— or —C(═O)OR⁶ wherein R⁶ is hydrogen, alkyl, alkenyl, alkynyl, cycloalkyl, cycloalkenyl, amino, thiol, aryl, heteroaryl, thioalkyl, thioaryl, thioalkoxy, a heteroaryl, or a heterocyclic, which may optionally be substituted. In aspects of the invention, the carboxyl groups are in an esterified form and may contain as an esterifying group lower alkyl groups. In particular aspects of the invention, —C(O)OR⁶ provides an ester or an amino acid derivative. An esterified form is also particularly referred to herein as a “carboxylic ester”. In aspects of the invention a “carboxyl” may be substituted, in particular substituted with alkyl which is optionally substituted with one or more of amino, amine, halo, alkylamino, aryl, carboxyl, or a heterocyclic. Examples of carboxyl groups are methoxycarbonyl, butoxycarbonyl, tert.alkoxycarbonyl such as tert.butoxycarbonyl, arylmethyoxycarbonyl having one or two aryl radicals including without limitation phenyl optionally substituted by for example lower alkyl, lower alkoxy, hydroxyl, halo, and/or nitro, such as benzyloxycarbonyl, methoxybenxyloxycarbonyl, diphenylmethoxycarbonyl, 2-bromoethoxycarbonyl, 2-iodoethoxycarbonyltertbutylcarbonyl, 4-nitrobenzyloxycarbonyl, diphenylmethoxy-carbonyl, benzhydroxycarbonyl, di-(4-methoxyphenyl-methoxycarbonyl, 2-bromoethoxycarbonyl, 2-iodoethoxycarbonyl, 2-trimethylsilylethoxycarbonyl, or 2-triphenylsilylethoxycarbonyl. Additional carboxyl groups in esterified form are silyloxycarbonyl groups including organic silyloxycarbonyl. In aspects of the invention, the carboxyl group may be an alkoxy carbonyl, in particular methoxy carbonyl, ethoxy carbonyl, isopropOxy carbonyl, t-butoxycarbonyl, t-pentyloxycarbonyl, or heptyloxy carbonyl, especially methoxy carbonyl or ethoxy carbonyl.

As used herein, the term “carbamoyl”, alone or in combination, refers to amino, monoalkylamino, dialkylamino, monocycloalkylamino, alkylcycloalkylamino, and dicycloalkylamino radicals, attached to one of two unshared bonds in a carbonyl group.

As used herein, the term “carboxamide” refers to the group —CONH—.

As used herein, the term “nitro” means —NO₂—.

As used herein, the term “acyl”, alone or in combination, means a carbonyl or thiocarbonyl group bonded to a radical selected from, for example, optionally substituted, hydrido, alkyl (e.g. haloalkyl), alkenyl, alkynyl, alkoxy (“acyloxy” including acetyloxy, butyryloxy, iso-valeryloxy, phenylacetyloxy, benzoyloxy, p-methoxybenzoyloxy, and substituted acyloxy such as alkoxyalkyl and haloalkoxy), aryl, halo, heterocyclyl, heteroaryl, sulfinyl (e.g. alkylsulfinylalkyl), sulfonyl (e.g. alkylsulfonylalkyl), cycloalkyl, cycloalkenyl, thioalkyl, thioaryl, amino (e.g alkylamino or dialkylamino), and aralkoxy. Illustrative examples of “acyl” radicals are formyl, acetyl, 2-chloroacetyl, 2-bromacetyl, benzoyl, trifluoroacetyl, phthaloyl, malonyl, nicotinyl, and the like.

In aspects of the invention, “acyl” refers to a group —C(O)R⁷, where R⁷ is hydrogen, alkyl, cycloalkyl, cycloheteroalkyl, aryl, arylalkyl, heteroalkyl, heteroaryl, and heteroarylalkyl. Examples include, but are not limited to formyl, acetyl, cyclohexylcarbonyl, cyclohexylmethylcarbonyl, benzoyl, benzylcarbonyl and the like.

As used herein, “ureido” refers to the group “—NHCONH—”. A ureido radical includes an alkylureido comprising a ureido substituted with an alkyl, in particular a lower alkyl attached to the terminal nitrogen of the ureido group. Examples of an alkylureido include without limitation N′-methylureido, N′-ethylureido, N′-n-propylureido, N′-i-propylureido and the like. A ureido radical also includes a N′,N′-dialkylureido group containing a radical —NHCON where the terminal nitrogen is attached to two optionally substituted radicals including alkyl, aryl, heterocylic, and heteroaryl.

The terms used herein for radicals including “alkyl”, “alkoxy”, “alkenyl”, “alkynyl”, “hydroxyl” etc. refer to both substituted and substituted radicals. The term “substituted,” as used herein, means that any one or more moiety on a designated atom (e.g., hydrogen) is replaced with a selection from a group disclosed herein, provided that the designated atom's normal valency is not exceeded, and that the substitution results in a stable compound. Combinations of substituents and/or radicals are permissible only if such combinations result in stable compounds. “Stable compound” refers to a compound that is sufficiently robust to survive isolation to a useful degree of purity from a reaction mixture, and formulation into an efficacious therapeutic agent.

A functional group or ring of a salt of a compound of the Formula I may be modified with, or a radical in such compound may be substituted with one or more groups or substituents apparent to a person skilled in the art including without limitation alkyl, alkoxy, alkenyl, alkynyl, alkanoyl, alkylene, alkenylene, hydroxyalkyl, haloalkyl, haloalkylene, haloalkenyl, alkoxy, alkenyloxy, alkenyloxyalkyl, alkoxyalkyl, aryl, alkylaryl, haloalkoxy, haloalkenyloxy, heterocyclic, heteroaryl, sulfonyl, alkylsulfonyl, sulfinyl, sulfenyl, alkylsulfinyl, aralkyl, heteroaralkyl, cycloalkyl, cycloalkenyl, cycloalkoxy, cycloalkenyloxy, amino, oxy, halo, azido, thio, ═O, ═S, cyano, hydroxyl, phosphonato, phosphinato, thioalkyl, alkylamino, arylamino, arylsulfonyl, alkylcarbonyl, arylcarbonyl, heteroarylcarbonyl, heteroarylsulfinyl, heteroarylsulfony, heteroarylamino, heteroaryloxy, heteroaryloxylalkyl, arylacetamidoyl, aryloxy, aroyl, aralkanoyl, aralkoxy, aryloxyalkyl, haloaryloxyalkyl, heteroaroyl, heteroaralkanoyl, heteroaralkoxy, heteroaralkoxyalkyl, thioaryl, arylthioalkyl, alkoxyalkyl, and acyl groups. These groups or substitutents may themselves be substituted.

Derivative groups that may be used to modify compounds of the Formula I can also be found in U.S. Patent Application No. 20030176437.

A chemical substituent is “pendant” from a radical if it is bound to an atom of the radical. In this context, the substituent can be pending from a carbon atom of a radical, a carbon atom connected to a carbon atom of the radical by a chain extender, or a heteroatom of the radical. The term “fused” means that a second ring is present (i.e., attached or formed) by having two adjacent atoms in common or shared with the first ring.

A “dosage form” refers to a composition or device comprising an acid addition salt of a compound of the formula I and pharmaceutically acceptable carrier(s), excipient(s), or vehicles. A dosage form may be an immediate release dosage form or a sustained release dosage form.

An “immediate release dosage form” refers to a dosage form which does not include a component for sustained release i.e., a component for slowing disintegration or dissolution of an active compound. These dosage forms generally rely on the composition of the drug matrix to effect the rapid release of the active ingredient agent.

By “sustained release dosage form” is meant a dosage form that releases active compound for many hours. In an aspect, a sustained dosage form includes a component for slowing disintegration or dissolution of the active compound. A dosage form may be a sustained release formulation, engineered with or without an initial delay period. Sustained release dosage forms may continuously release drug for sustained periods of at least about 4 hour's or more, about 6 hours or more, about 8 hours or more, about 12 hours or more, about 15 hours or more, or about 20 hours to 24 hours. A sustained release dosage form can be formulated into a variety of forms, including tablets, lozenges, gelcaps, buccal patches, suspensions, solutions, gels, etc. In aspects of the invention the sustained release form results in administration of a minimum number of daily doses.

A “disease” that can be treated and/or prevented using a salt, formulation, dosage form, composition and method of the invention includes a condition associated with or requiring modulation of one or more of inflammation (e.g. neuroinflammation), signaling pathways involved in inflammation (e.g., neuroinflammation), cell signaling molecule production, activation of glia or glial activation pathways and responses, proinflammatory cytokines or chemokines (e.g., interleukin (IL) or tumor necrosis factor (TNF), activation of astrocytes or astrocyte activation pathways and responses, activation of microglia or microglial activation pathways and responses, oxidative stress-related responses such as nitric oxide synthase production and nitric oxide accumulation, acute phase proteins, components of the complement cascade, loss or reduction of synaptic function, loss of synaptophysin and/or PSD-95; protein kinase activity (e.g., death associated protein kinase (DAPK) activity), amyloid β deposition of amyloid plaques, behavioral deficits, cell damage (e.g., neuronal cell damage), and cell death (e.g., neuronal cell death). In particular a disease is a dementing disorder, a neurodegenerative disorder, a pain disorder, a CNS demyelinating disorder, an autoimmune disorder, or a peripheral inflammatory disease.

A disease may be characterized by an inflammatory process due to the presence of macrophages activated by an amyloidogenic protein or peptide. Thus, a method of the invention may involve inhibiting macrophage activation and/or inhibiting an inflammatory process. A method may comprise decreasing, slowing, ameliorating, or reversing the course or degree of macrophage invasion or inflammation in a patient.

Examples of diseases that can be treated and/or prevented using the salts, formulations, dosage forms, compositions and methods of the invention, include Alzheimer's disease and related disorders, presenile and senile forms; amyloid angiopathy; mild cognitive impairment; Alzheimer's disease-related dementia (e.g., vascular dementia or Alzheimer dementia); AIDS related dementia, tauopathies (e.g., argyrophilic grain dementia, corticobasal degeneration, dementia pugilistica, diffuse neurofibrillary tangles with calcification, frontotemporal dementia with parkinsonism, Prion-related disease, Hallervorden-Spatz disease, myotonic dystrophy, Niemann-Pick disease type C, non-Guamanian Motor Neuron disease with neurofibrillary tangles, Pick's disease, postencephalitic parkinsonism, cerebral amyloid angiopathy, progressive subcortical gliosis, progressive supranuclear palsy, subacute sclerosing panencephalitis, and tangle only dementia), alpha-synucleinopathy (e.g., dementia with Lewy bodies, multiple system atrophy with glial cytoplasmic inclusions), multiple system atrophies, Shy-Drager syndrome, spinocerebellar ataxia (e.g., DRPLA or Machado-Joseph Disease); striatonigral degeneration, olivopontocerebellar atrophy, neurodegeneration with brain iron accumulation type I, olfactory dysfunction, and amyotrophic lateral sclerosis); Parkinson's disease (e.g., familial or non-familial); Amyotrophic Lateral Sclerosis; Spastic paraplegia (e.g., associated with defective function of chaperones and/or triple A proteins); Huntington's Disease, spinocerebellar ataxia, Freidrich's Ataxia; cerebrovascular diseases including stroke, hypoxia, ischemia, infarction, intracerebral hemorrhage; traumatic brain injury; Down's syndrome; head trauma with post-traumatic accumulation of amyloid beta peptide; Familial British Dementia; Familial Danish Dementia; Presenile Dementia with Spastic Ataxia; Cerebral Amyloid Angiopathy, British Type; Presenile Dementia With Spastic Ataxia Cerebral Amyloid Angiopathy, Danish Type; Familial encephalopathy with neuroserpin inclusion bodies (FENIB); Amyloid Polyneuropathy (e.g., senile amyloid polyneuropathy or systemic Amyloidosis); Inclusion Body myositis due to amyloid beta peptide; Familial and Finnish Type Amyloidosis; Systemic amyloidosis associated with multiple myeloma; Familial Mediterranean Fever; multiple sclerosis, optic neuritis; Guillain-Barre Syndrome; chronic inflammatory demyelinating polyneuropathy; chronic infections and inflammations; acute disseminated encephalomyelitis (ADEM); autoimmune inner ear disease (AIED); diabetes; myocardial ischemia and other cardiovascular disorders; pancreatitis; gout; inflammatory bowel disease; ulcerative colitis, Crohn's disease, rheumatoid arthritis, osteoarthritis; artheriosclerosis, inflammatory aortic aneurysm; asthma; adult respiratory distress syndrome; restenosis; ischemia/reperfusion injury; glomerulonephritis; sacoidosis cancer; restenosis; rheumatic fever; systemic lupus erythematosus; Reiter's syndrome; psoriatic arthritis; ankylosing spondylitis; coxarthritis; pelvic inflammatory disease; osteomyelitis; adhesive capsulitis; oligoarthritis; periarthritis; polyarthritis; psoriasis; Still's disease; synovitis; inflammatory dermatosis; wound healing.

In aspects of the invention, the disease is Alzheimer's disease, vascular dementia, dementia associated with Parkinson's disease, visuospatial deficits, Williams syndrome, encephalitis, meningitis, fetal alcohol syndrome, Korsakoffs syndrome, anoxic brain injury, cardiopulmonary resuscitation injuries, diabetes, Sjogren's syndrome, strokes, ocular diseases such as cataracts and macular degeneration, sleep disorders, and cognitive impairments caused by high cholesterol levels.

In aspects of the invention, a salt, formulation, dosage form, composition, and method disclosed herein may be utilized to prevent and/or treat a disease involving neuroinflammation (i.e., neuroinflammatory disease). Neuroinflammation is a characteristic feature of disease pathology and progression in a diverse array of neurodegenerative disorders that are increasing in their societal impact (for a recent review, see, e.g., Prusiner, S. B. (2001) New Engl. J. Med. 344, 1516-1526). These neuroinflammation-related disorders include Alzheimer's disease (AD), amyotrophic lateral sclerosis, autoimmune disorders, priori diseases, stroke and traumatic brain injury. Neuroinflammation is brought about by glial cell (e.g., astrocytes and microglia) activation, which normally serves a beneficial role as part of an organism's homeostatic response to injury or developmental change. However, disregulation of this process through chronic or excessive activation of glia contributes to the disease process through the increased production of proinflammatory cytokines and chemokines, oxidative stress-related enzymes, acute phase proteins, and various components of the complement cascades. (See, e.g., Akiyama et al., (2000) Neurobiol. Aging 21, 383-421). The direct linkage of glial activation to pathology that is a hallmark of disease underscores the importance of understanding the signal transduction pathways that mediate these critical glial cellular responses and the discovery of cell permeable ligands that can modulate these disease relevant pathways.

In certain selected aspects of the invention, the disease is a neurodegenerative disease or neurodegenerative disorder including such diseases and impairments as Alzheimer's disease, dementia, MCI, Huntington's disease, Parkinson's disease, amyotrophic lateral sclerosis, and other similar diseases and disorders disclosed herein.

For Alzheimer's disease (AD) in particular, the deposition of β-amyloid (Aβ) and neurofibrillary tangles are associated with glial activation, neuronal loss and cognitive decline. On a molecular level, Alzheimer's disease is characterized by; increased expression of nitric oxide synthase (NOS) in glial cells surrounding amyloid plaques; neuropathological evidence of peroxynitrite-mediated neuronal damage; and nitric oxide (NO) overproduction involved in Aβ-induced brain dysfunction. NOSH (iNOS) is induced as part of the glial activation response and is an oxidative stress-related enzyme that generates NO. When NO is present in high levels along with superoxide, the highly reactive NO-derived molecule peroxynitrite is generated, leading to neuronal cell death. The pro-inflammatory cytokine IL-1β is also overexpressed in activated glia in AD brain and polymorphisms in IL-1β genes are associated with an increased risk of early onset sporadic AD (See, e.g., Du et al., (2000) Neurology 55, 480-483). IL-1β can also influence amyloid plaque development and is involved in additional glial inflammatory and neuronal dysfunction responses (See, e.g., Griffin, et al., (1998) Brain Pathol. 8, 65-72; and Sheng, et al., (1996) Neurobiol. Aging 17, 761-766). Therefore, because glial activation and specific glial products are associated with neurodegenerative disorders (e.g., Alzheimer's disease), the compounds, dosage forms and compositions disclosed herein that are capable of modulating cell signaling pathways (e.g., glial activation pathways) will have particular application in the treatment and prevention of inflammatory disease.

In aspects of the invention, a salt, dosage form, formulation, composition, or method disclosed herein may be utilized to prevent and/or treat a disease involving disregulation of protein kinase signaling. Disregulation of protein kinase signaling often accompanies disregulation of cell signaling pathways (e.g., glial cell activation pathways). Protein kinases are a large family of proteins that play a central role in regulating a number of cellular functions including cell growth, differentiation and death. There are thought to be more than 500 protein kinases and 130 protein phosphatases exerting tight control on protein phosphorylation. Each protein kinase transfers the γ-phosphate of ATP to a specific residue(s) of a protein substrate. Protein kinases can be further categorized as tyrosine, serine/threonine or dual specific based on acceptor residue. Examples of serine/threonine kinases include MAP kinase, MAPK kinase (MEK), Akt/PKB, Jun kinase (INK), CDKs, protein kinase A (PRA), protein kinase C (PKC), and calmodulin (CaM)-dependent kinases (CaMKs). Disregulated protein kinase activity (e.g., hyper- or hypo-active) leads to abnormal protein phosphorylation, underlying a great number of diseases including diabetes, rheumatoid arthritis, inflammation, hypertension, and proliferative diseases such as cancer. Therefore, because aberrant kinase activity is associated with inflammatory disease (e.g., neurodegenerative disorders like Alzheimer's disease), the compounds and compositions that are disclosed herein that are capable of modulating kinases involved in cell signaling pathways will have particular application for treatment and prevention of inflammatory disease.

Diseases that may also be treated and/or prevented according to the invention include Demyelinating Diseases. “Demyelinating Diseases” refers to diseases in which myelin is the primary target. These diseases can be divided into two groups: Acquired Diseases and Hereditary Metabolic Disorders. Acquired Demyelinating Diseases include Multiple sclerosis (MS) including its alternating relapsing/remitting phases, Hereditary Metabolic Disorders includes the leukodystrophies such as metachromatic leukodystrophy, Refsum's disease, adrenoleukodystrophy, Krabbe's disease, phenylketonuria, Canavan disease, Pelizaeus-Merzbacher disease and Alexander's disease.

Diseases that may also be treated and/or prevented according to the invention include “Demyelinating Conditions”. The term refers to conditions that result in deficient myelination. Such conditions include, but are not limited to, Spinal Cord Injury, Traumatic Brain Injury and Stroke.

The term “Spinal Cord Injury (SCI)” refers to an injury to the spinal cord which results in loss of function such as mobility or feeling.

The term “Traumatic Brain Injury (TBI)” refers to an injury which results in damage to the brain. A head injury may be a closed head injury or penetrating head injury. A closed head injury may occur when the head is hit by a blunt object causing the brain to interact with the hard bony surface inside the skull. A closed head injury may also occur without direct external trauma to the head if the brain undergoes a rapid forward or backward movement, (e.g. whiplash). A penetrating head injury may occur when a fast moving object such as a bullet pierces the skull. A closed or penetrating head injury may result in localized and widespread, or diffuse, damage to the brain which may manifest as memory loss, emotional disturbances, motor difficulties, including paralysis, damage to the senses, and death. The term also includes secondary damage that follows an injury including swelling and fluid buildup, and the accumulation of substances toxic to surrounding neurons such as the neurotransmitter glutamate.

The term “Stroke” refers to a sudden loss of brain function caused by the interruption of the flow of blood to the brain (an ischemic stroke) or the rupture of blood vessels in the brain (a hemorrhagic stroke). The interruption of the blood flow or the rupture of blood vessels causes neurons in the affected area to die. The term also includes stroke rehabilitation which refers to the intervention resulting in the full or partial recovery of functions that have been lost due to stroke.

A pain disorder may also be treated and/or prevented according to the invention. A “pain disorder” refers to a disorder or condition involving pain and includes without limitation acute pain, persistent pain, chronic pain, inflammatory pain, neuropathic pain, neurogenic pain, and chemokine-induced pain. In aspects of the invention, a pain disorder includes without limitation pain resulting from soft tissue and peripheral damage such as acute trauma; complex regional pain syndrome also referred to as reflex sympathetic dystrophy; postherpetic neuralgia, occipital neuralgia, trigeminal neuralgia, segmental or intercostal neuralgia and other neuralgias; pain associated with osteoarthritis and rheumatoid arthritis; musculo-skeletal pain such as pain associated with strains, sprains and trauma such as broken bones; spinal pain, central nervous system pain such as pain due to spinal cord or brain stem damage; lower back pain, sciatica, dental pain, myofascial pain syndromes, episiotomy pain, gout pain, and pain resulting from burns; deep and visceral pain, such as heart pain; muscle pain, eye pain, inflammatory pain, orofacial pain, for example, odontalgia; abdominal pain, and gynecological pain, for example, dysmenorrhoea, labour pain and pain associated with endometriosis; somatogenic pain; pain associated with nerve and root damage, such as pain associated with peripheral nerve disorders, for example, nerve entrapment, brachial plexus avulsions, and peripheral neuropathies; pain associated with limb amputation, tic douloureux, neuroma, or vasculitis; diabetic neuropathy, chemotherapy-induced-neuropathy, acute herpetic and postherpetic neuralgia; atypical facial pain, nerve root damage, neuropathic lower back pain, HIV related neuropathic pain, cancer related neuropathic pain, diabetes related neuropathic pain and arachnoiditis, trigeminal neuralgia, occipital neuralgia, segmental or intercostal neuralgia, HIV related neuralgias and AIDS related neuralgias and other neuralgias; allodynia, hyperalgesia, idiopathic pain, pain caused by chemotherapy; occipital neuralgia, psychogenic pain, brachial plexus avulsion, pain associated with restless legs syndrome; pain associated with gallstones; pain caused by chronic alcoholism or hypothyroidism or uremia or vitamin deficiencies; neuropathic and non-neuropathic pain associated with carcinoma, often referred to as cancer pain, phantom limb pain, functional abdominal pain, headache, including migraine with aura, migraine without aura and other vascular headaches, acute or chronic tension headache, sinus headache and cluster headache; temperomandibular pain and maxillary sinus pain; pain resulting from ankylosing spondylitis and gout; pain caused by increased bladder contractions; pain associated with gastrointestinal (GI) disorders, disorders caused by helicobacter pylori and diseases of the GI tract such as gastritis, proctitis, gastroduodenal ulcers, peptic ulcers, dyspepsia, disorders associated with the neuronal control of viscera, ulcerative colitis, chronic pancreatitis, Crohn's disease and emesis; post operative pain, scar pain, and chronic non-neuropathic pain such as pain associated with HIV, anthralgia and myalgia, vasculitis and fibromyalgia.

The term “Neuropathic pain” refers to pain initiated or caused by a primary lesion or dysfunction in the nervous system. The term “Neuropathic pain” refers to pain initiated or caused by a primary lesion or dysfunction in the nervous system. For the purpose of this invention included under this heading or to be treated as synonymous is “Neurogenic Pain” which is defined as pain initiated or caused by a primary lesion, dysfunction or transitory perturbation in the peripheral or central nervous system. In aspects, the uses of the present invention include central or peripheral neuropathic pain or neurogenic pain. In other aspects, neuropathic pain includes the pain caused by either mononeuropathy or polyneuropathy. Neuropathic pain also includes Chemokine-Induced Pain.

“Peripheral neuropathic pain” refers to a pain initiated or caused by a primary lesion or dysfunction in the peripheral nervous system and “peripheral neurogenic pain” refers to a pain initiated or caused by a primary lesion, dysfunction or transitory perturbation in the peripheral nervous system. A peripheral neuropathic pain can be allodynia (i.e., a pain due to a stimulus which does not normally provoke pain); causalgia (i.e., a syndrome of sustained burning pain, allodynia and hyperpathia after a traumatic nerve lesion, often combined with vasomotor and sudomotor dysfunction and later trophic changes); hyperalgesia (i.e., an increased response to a stimulus which is normally painful); hyperesthesia (i.e., increased sensitivity to stimulation, excluding the senses); hyperpathia (i.e., a painful syndrome characterized by an abnormally painful reaction to a stimulus, especially a repetitive stimulus, as well as an increased threshold); neuritis (i.e., inflammation of a nerve or nerves); or neuropathy (i.e., a disturbance of function or pathological change in a nerve). [See IASP, Classification of chronic pain, 2nd Edition, IASP Press (2002), for detailed definitions of these categories of neuropathic pain and neurogenic pain).

Exemplary types of neuropathic pain include infective (e.g., post herpetic neuralgia and HIV neuropathy), metabolic (e.g., diabetic neuropathy and Fabry's disease), toxic (e.g., from lead or chemotherapy), traumatic/stretch injury (e.g.; post incisional, trauma, phantom limb pain, and reflex sympathetic dystrophy/complex regional pain syndrome/causalgia), and idiopathic (e.g., trigeminal neuralgia/tic douloureux).

Particular examples of Neuropathic Pain include post-herpetic neuralgia, painful diabetic neuropathy, phantom limb pain, central post-stroke pain, HIV neuropathy, Fabry's disease, peripheral neuropathy, trigeminal neuralgia, post incisional neuropathic pain, phantom limb pain, reflex sympathetic dystrophy, causalgia, anesthesia dolorosa, intercoastal neuralgia, post-traumatic localized pain, atypical facial neuralgia pain after tooth extraction and the like, complex regional pain syndrome, neuropathic pain caused by trauma, lead, or chemotherapy, cancer pain resistant to narcotic analgesics such as morphine.

Treatment of neuropathic pain may be defined as administration of a therapeutic dose of a salt of a compound of the formula I to reduce and preferably eliminate pain that results from nerve injury. Treatment of nerve injury may be defined as administration of a therapeutic dose of a salt of a compound of the formula Ito ameliorate injury and to increase the rate of recovery. An increased rate of recovery is defined as a reduction of indications of pain from peripheral nerve injury, such as thermal hyperalgesia and mechanical allodynia, more quickly than would be accomplished without pharmacological or other medical intervention.

“Chemokine-Induced Pain” refers to pain that occurs in response, in whole or in part, to chemokines, in particular pro-inflammatory cytokines (e.g. fractalkine, CCL2, and CCL5). An example of chemokine-induced pain is arthritic pain.

### Salts

The present invention relates to stable and substantially purified synthetic pharmaceutically acceptable acid addition salts of pyridazine compounds of the formula I, wherein R¹⁰ is hydrogen, hydroxyl, alkyl, alkoxy, alkenyl, alkynyl, alkylene, aryl, heteroaryl, sulfonyl, sulfinyl, sulfenyl, amino, imino, azido, thiol, thioalkyl, thioalkoxy, thioaryl, nitro, cyano, halo, ═O, ═S, carboxyl, carbonyl, carbamoyl, carboxamide, or phosphonate, and is alkyl, alkoxy, alkenyl, alkynyl, alkylene, alkenylene, alkenyloxy, aryl, aryloxy, arylalkoxy, aroyl, cycloalkyl, cycloalkenyl, cycloalkynyl, cycloalkoxy, heteroaryl, heterocyclic, acyl, acyloxy, sulfonyl, sulfinyl, sulfenyl, amino, imino, azido, thiol, thioalkyl, thioalkoxy, thioaryl, nitro, cyano, halo, silyl, silyloxy, silylthio, ═O, ═S, phosphonate, carboxyl, carbonyl, carbamoyl, carboxamide, or ureido.

Aspects of the invention utilize a compound of the formula I wherein R¹⁰ is hydrogen, hydroxyl, alkyl, alkoxy, alkenyl, alkynyl, alkylene, aryl, heteroaryl, sulfonyl, sulfinyl, sulfenyl, amino, imino, azido, thiol, thioalkyl, thioalkoxy, thioaryl, nitro, cyano, halo, ═O, ═S, carboxyl, carbonyl, carbamoyl, carboxamide, or phosphonate, and R¹¹ is alkyl, alkoxy, alkenyl, alkynyl, alkylene, alkenylene, alkenyloxy, aryl, aryloxy, arylalkoxy, aroyl, heteroaryl, acyl, acyloxy, sulfonyl, sulfinyl, sulfenyl, amino, imino, azido, thiol, thioalkyl, thioalkoxy, thioaryl, nitro, cyano, halo, ═O, ═S, carboxyl, carbonyl, carbamoyl, carboxamide, or ureido.

In an aspect, R¹⁰ is hydrogen, hydroxyl, C₁-C₆ alkyl, C₂-C₆ alkenyl, C₂-C₆ alkynyl, C₁-C₆alkoxy, C₆-C₁₀aryl, C₃-C₁₀heteroaryl, sulfonyl, sulfinyl, sulfenyl, amino, thiol, thio C₁-C₆alkyl, thio C₁-C₆alkoxy, nitro, halo, ═O, ═S, or carboxyl, and R¹¹ is C₁-C₆ alkyl, C₂-C₆ alkenyl, C₂-C₆ alkynyl, C₁-C₆alkoxy; C₃-C₁₀heteroaryl, sulfonyl, sulfinyl, sulfenyl, amino, thiol, thio C₁-C₆ alkyl, thio C₁-C₆ alkoxy, nitro, cyano, halo, ═O, ═S, or carboxyl, carbonyl, carbamoyl, carboxamide, or ureido.

In further aspects of the invention, pure, in particular, substantially pure, compounds of the formula I are provided wherein R¹⁰ is hydrogen, hydroxyl, alkyl, alkoxy, alkenyl, aryl, heteroaryl, sulfonyl, sulfinyl, sulfenyl, amino, thiol, thioalkyl, thioalkoxy, nitro, halo, ═O, ═S, or carboxyl; and R¹¹ is alkyl, alkenyl, alkynyl, alkylene, alkenylene, alkoxy, alkenyloxy, aryl, aryloxy, arylalkoxy, aroyl, heteroaryl; or an isomer, a pharmaceutically acceptable salt, or derivative thereof.

In further aspects of the invention, pure, in particular, substantially pure, compounds of the formula I are provided wherein R¹⁰ is hydrogen, hydroxyl, C₁-C₆ alkyl, C₁-C₆ alkoxy, C₁-C₆ alkenyl, C₆-C₁₀ aryl, C₃-C₁₀ heteroaryl, sulfonyl, sulfinyl, sulfenyl, amino, thiol, thio C₁-C₆alkyl, thio C₁-C₆alkoxy, nitro, halo, ═O, ═S, or carboxyl; and R^{11 is C}₁-C₆alkyl, C₁-C₆alkenyl, C₁-C₆alkynyl, C₁-C₆alkylene, C₂-C₈alkenylene, C₁-C₆alkoxy, C₂-C₁₀alkenyloxy, C₆-C₁aryl, C₆-C₁₀aryloxy, C₆-C₁₀aryl C₁-C₆alkoxy, C₆-C₁₀aryl, C₃-C₁₀ heteroaryl; or an isomer, a pharmaceutically acceptable salt, or derivative thereof.

In aspects of the invention, a compound of the formula I is used wherein R¹⁰ is hydrogen, alkyl, alkoxy, sulfonyl, sulfinyl, halo, thiol, or carboxyl, and R¹¹ is alkyl, alkenyl, alkoxy, alkenyloxy, aryl, heteroaryl, amino, imino, azido, thiol, thioalkyl, thioalkoxy, nitro, cyano, halo, ═O, ═S, carboxyl, carbonyl, carbamoyl, or carboxamide; or an isomer or a pharmaceutically acceptable salt thereof.

In aspects of the invention, a compound of the formula I is used wherein R¹⁰ is hydrogen; C₁-C₆alkyl, C₁-C₆alkoxy, sulfonyl, sulfinyl, halo, thiol, or carboxyl, and R¹¹ is C₁-C₆alkyl, C₁-C₆alkenyl, C₁-C₆alkoxy, C₂-C₁₀alkenyloxy, C₆-C₁₀aryl, C₃-C₁₀heteroaryl, amino, imino, azido, thiol, thio C₁-C₆alkyl, thio C₁-C₆alkoxy, nitro, cyano, halo, ═O, ═S, carboxyl, carbonyl, carbamoyl, or carboxamide; or an isomer or a pharmaceutically acceptable salt thereof.

In aspects of the invention, R¹⁰ is hydrogen, alkyl, alkoxy, amino, thiol, halo, ═O, ═S, phosphonate, or carboxyl, more particularly R¹⁰ is hydrogen or alkyl.

In aspects of the invention, R¹⁰ is hydrogen, C₁-C₆alkyl, C₁-C₆alkoxy, amino, thiol, halo, ═O, ═S; phosphonate, or carboxyl, more particularly R¹⁰ is hydrogen or C₁-C₆alkyl.

In aspects of the invention, R¹¹ is alkyl, alkoxy, alkenyl, aryl, or heteroaryl, more particularly alkyl or heteroaryl.

In aspects of the invention, R¹¹ is C₁-C₆alkyl, C₁-C₆alkoxy, C₁-C₆alkenyl, C₆-C₁₀aryl, or C₃-C₁₀heteroaryl, more particularly C₁-C₆alkyl or C₃-C₁₀heteroaryl.

In an embodiment of the invention, R¹¹ is alkyl or heteroaryl. In an embodiment of the invention, one of R¹⁰ and R¹¹ in a compound of the formula I is a heteroaryl, in particular an unsaturated 5 to 6 membered heteromonocyclyl group containing 1 to 4 nitrogen atoms, more particularly pyridinyl, and the other of R¹⁰ and R¹¹ is hydrogen.

In another embodiment of the invention a compound of the formula I is employed wherein R¹¹ is hydrogen, halo, optionally substituted alkyl, pyridinyl, piperidinyl, morpholinyl, piperazinyl, or phenyl.

In other aspects of the invention, a compound of the formula I is employed wherein R¹¹ is alkyl, aryl, or an unsaturated 5 to 6 membered heteromonocyclyl group containing 1 to 4 nitrogen atoms. In particular aspects, R¹¹ is substituted or unsubstituted alkyl; phenyl, benzyl, or pyridinyl, more particularly R¹¹ is alkyl.

In an embodiment of the invention, one of R¹⁰ and R¹¹ in a compound of the formula I is aryl or substituted aryl, in particular phenyl or benzyl, and the other of R¹⁰ and R¹¹ is hydrogen.

In another embodiment of the invention, one of R¹⁰ and R¹¹ in a compound of the formula I is alkyl, in particular C₁-C₆ alkyl and the other of R¹⁰ and R¹¹ is hydrogen.

In a further embodiment of the invention, one of R¹⁰ and R¹¹ in a compound of the formula I is alkyl, in particular C₁-C₆ alkyl and the other of R¹⁰ and R¹¹ is hydrogen.

In a particular embodiment of the invention, R¹¹ in a compound of the formula I is alkyl, in particular C₁-C₆ alkyl and R¹⁰ is hydrogen.

A pharmaceutically acceptable acid addition salt of a compound of the formula I includes salts formed with inorganic acids, such as hydrochloric and hydrobromic acids, and organic acids such as acetic acid, citric acid, maleic acid, and the alkane- and arene-sulfonic acids such as methanesulfonic acid and benezenesulfonic acid. When there are two acidic groups present, a pharmaceutically acceptable salt according to the invention may be a mono-acid-mono-salt or a di-salt; and similarly where there are more than two acidic groups present, some or all of such groups can be salified.

In aspects of the invention, the pharmaceutically acceptable acid addition salts according to the invention are salts formed with inorganic acids. In particular aspects, the pharmaceutically acceptable acid addition salts according to the invention are halide salts, more particularly chloride or bromide salts of a compound of the formula I.

In aspects of the invention, pharmaceutically acceptable acid addition salts, in particular inorganic acid salts, more particularly halide salts, most particularly chloride salts of compounds of the formula I, are provided having enhanced stability properties as compared to the parent compound or lead compound, and/or having properties which may enable them to dissolve and target faster.

In aspects of the invention, the counter-cation of a halide salt may be an alkali metal (e.g. Li, Na, or K), or preferably, hydrogen. In an embodiment, the invention provides a stable and substantially purified hydrochloride salt of a compound of the formula I, in particular a di-hydrochloride salt of a compound of the formula I. A hydrochloride salt may be further characterized by having enhanced stability properties as compared to the parent or lead compound, and/or enhanced dissolution rates.

In an embodiment of the invention, a hydrochloride salt of a compound of the formula I is provided that has greater thermal stability than the parent compound (e.g. it is more stable than the parent or lead compound when exposed to atmospheric oxygen).

An aspect of the invention provides a salt according to the invention in a non-crystalline form.

Another aspect of the invention relates to obtaining certain acid addition salts, in particular halide salts, more particularly chloride or bromide salts, most particularly hydrochloride and hydrobromide salts of a compound of the formula I of sufficient quality to determine the three dimensional (tertiary) structure of the compounds by X-ray diffraction methods. Accordingly, the invention provides crystals of sufficient quality to obtain a determination of the three-dimensional structure of the acid addition salts, in particular halide salts, more particularly chloride or bromide salts, most particularly hydrochloride and hydrobromide salts of a compound of the formula I, to high resolution. Therefore, the present invention provides stable crystalline acid addition salts, in particular halide salts, more particularly chloride or bromide salts, most particularly hydrochloride and hydrobromide salts of a compound of the formula I. In particular, the invention relates to a stable crystalline chloride or bromide salt of a compound of the formula I comprising molecules of chloride or bromide salts of a compound of the formula I in a unit cell held together by hydrogen bond interactions. In embodiments, the crystalline chloride and bromide salts comprises molecules of hydrochloride or hydrobromide salts of a compound of the formula I.

In another embodiment, the present invention provides a crystalline chloride or bromide salt of a Compound of the formula I. A crystalline chloride or bromide salt of a compound of the formula I may comprise molecules of chloride or bromide salts of a compound of the formula I in a unit cell held together by hydrogen bond interactions.

A crystal may take any crystal symmetry form based on the type of salt molecule, the hydrogen bond interactions, and/or the space group. The symmetry form is defined by the “unit cell” which is the basic parallelepiped that repeats in each direction to form the crystal lattice. The term “space group” refers to the arrangement of symmetry elements of a crystal. It will also be appreciated that crystalline salts, in particular crystalline chloride or bromide salts (more particularly hydrochloride or hydrobromide salts), of functional derivatives of compounds of the formula I may be prepared using the methods described herein, and the salts prepared by the methods are contemplated in the present invention.

An acid additional salt of a compound of the Formula I may include a pharmaceutically acceptable co-crystal. A pharmaceutically acceptable co-crystal includes a co-crystal that is suitable for use in contact with the tissues of a subject or patient without undue toxicity, irritation, allergic response and has the desired pharmacokinetic properties.

The term “co-crystal” refers to a crystalline material comprised of two or more unique solids at room temperature, each containing distinctive physical characteristics, such as structure, melting point, and heats of fusion. Co-crystals can be formed by an active pharmaceutical ingredient (API) and a co-crystal former either by hydrogen bonding or other non-covalent interactions, such as pi stacking and van der Waals interactions. An aspect of the invention provides for a co-crystal wherein the co-crystal former is a second API. In another aspect, the co-crystal former is not an API. In another aspect, the co-crystal comprises more than one co-crystal former. For example, two, three, four, five, or more co-crystal formers can be incorporated in a co-crystal with an API. Pharmaceutically acceptable co-crystals are described, for example, in “Pharmaceutical co-crystals,” Journal of Pharmaceutical Sciences, Volume 95 (3) Pages 499-516, 2006. The methods producing co-crystals are discussed in the United States Patent Application 20070026078.

The invention also relates to a pharmaceutically acceptable acid addition salt of a compound of the formula I in amorphous form.

In general, all physical forms of addition salts of compounds of the Formula I are intended to be within the scope of the present invention.

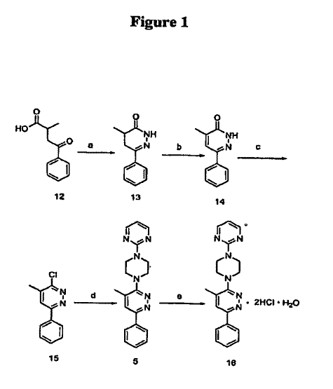

In a particular aspect of the invention, a pharmaceutically acceptable acid addition salt according to the invention is a halide salt of the pyridazine compound 4-methyl-6-phenyl-3-(4-pyrimidin-2-ylpiperazin-1-yl)pyridazine (5) shown in FIG. 1. In an embodiment, a pharmaceutically acceptable salt according to the invention is a chloride salt of 4-methyl-6-phenyl-3-(4-pyrimidin-2-ylpiperazin-1-yl)pyridazine (5) shown in FIG. 1. In a particular embodiment, a pharmaceutically acceptable salt is a hydrochloride salt of 4-methyl-6-phenyl-3-(4-pyrimidin-2-ylpiperazin-1-yl)pyridazine (5) shown in FIG. 1, more particularly a di-hydrochloride hydrate salt shown in FIG. 1 (i.e., 2-(4-(4-methyl-6-phenylpyridazin-3-yl)piperazin-1-ylpyrimidine dihydrochloride salt)(6).

In a particular embodiment, this invention provides a di-hydrochloride hydrate salt of a compound of the formula I, in particular 2-(4-(4-methyl-6-phenylpyridazin-3-yl)piperazin-1-yl)pyrimidine dihydrochloride salt (6) as shown in FIG. 1 characterized by enhanced solubility compared to a compound such as compound 1 in Table 1. This di-hydrochloride hydrate salt may be further characterized by one or more of the following: a yellow powder, having a purity of greater than about 90%, 92%, 93%, 94%, 95%, 96%, 97%, 98%, or 99%, molecular weight of 423.3395, soluble in DMSO, melting point of greater than 488K, acid dissociation constants (pKa) of 2.55, 1.46, 0.84 and −4.31 (calculated), logP of 2.29 determined from octanol/water partition coefficient, logS of 2.5 (experimental)/4.08 (calculated), and/or having an aqueous solubility at 37° C. of about 100 to 400 mg/ml, about 100 to 350 mg/ml, about 150 to 350 mg/ml, about 200 to 350 mg/ml, or about 300 to 350 mg/ml, in particular a solubility of greater than 322 mg/ml in water (2HCL.H₂O) salt).

In aspects, the invention relates to halide salts of a compound of the formula I in amorphous or crystalline form that have an enhanced resorption rate. In particular aspects the resorption rate is increased by a factor of at least 2, 3, 4 or 5 when compared to the parent compound.

### Preparation of Salts

Pyridazine compounds of the formula I may be prepared using reactions and methods generally known to the person of ordinary skill in the art. [See for example, US Published Application No. 2003-0176437, PCT Published Application No. WO 03/018563; and PCT Application No. PCT/US05/39541]. The reactions are performed in a solvent appropriate to the reagents and materials used and suitable for the reactions being effected. It will be understood by those skilled in the art of organic synthesis that the functionality present on the compounds should be consistent with the proposed reaction steps. This will sometimes require modification of the order of the synthetic steps or selection of one particular process scheme over another in order to obtain a desired compound of the invention. It will also be recognized that another major consideration in the development of a synthetic route is the selection of the protecting group used for protection of the reactive functional groups present in the compounds described in this invention. An authoritative account describing the many alternatives to the skilled artisan is Greene and Wuts (Protective Groups In Organic Synthesis, Wiley and Sons, 1991).

The starting materials and reagents used in preparing compounds of the formula I are either available from commercial suppliers or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J. Advanced Organic Chemistry, 4th ed., John Wiley and Sons, New York, N.Y.; and Larock: Comprehensive Organic Transformations, VCH Publishers, New York, 1989.

The starting materials, intermediates, and compounds of the formula I may be isolated and purified using conventional techniques, such as precipitation, filtration, distillation, crystallization, chromatography, and the like. The compounds of the formula I may be characterized using conventional methods; including physical constants and spectroscopic methods, in particular HPLC.

In an aspect, the invention provides a process for preparing a compound of the formula I wherein R¹¹ is hydrogen and R¹⁰ is an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, more particularly pyridinyl, which comprises reading a compound of the formula I wherein R¹⁰ is halo, in particular chloro, and R¹¹ is hydrogen, with boronic acid substituted with an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, more particularly pyridinyl, under suitable conditions to prepare a compound of the formula I wherein R¹¹ is hydrogen and R¹⁰ is an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, more particularly pyridinyl. In an embodiment, R¹⁰ is phenyl substituted with halo.

In another aspect, the invention provides a process for preparing a compound of the formula I wherein R¹¹ is hydrogen and R¹⁰ is a substituted aryl which comprises reacting a compound of the formula I wherein R¹⁰ is halo, in particular chloro, and R¹¹ is hydrogen, with a substituted aryl boronic acid under suitable conditions to prepare a compound of the formula I wherein R¹¹ is hydrogen and R¹⁰ is a substituted aryl. In an embodiment, R¹⁰ is phenyl substituted with halo.

In another aspect, the invention provides a process for preparing a compound of the formula I wherein R¹⁰ is hydrogen and R¹¹ is alkyl which comprises reacting a compound of the formula I wherein R¹¹ is halo, in particular chloro, and R¹⁰ is hydrogen, with an alkyl boronic acid under suitable conditions to prepare a compound of the formula I wherein R¹⁰ is hydrogen and R¹¹ is alkyl. In an embodiment, R¹¹ is lower alkyl, in particular methyl or ethyl, and a compound of the formula I wherein R¹¹ is chloro is reacted with lower alkyl boronic kid, in particular methyl or ethyl boronic acid under suitable conditions.

In another aspect, the invention provides a process for preparing a compound of the formula I wherein R¹⁰ is hydrogen and R¹¹ is an alkyl which comprises reacting a pyridazine substituted at the C3 position with halo (e.g., chloro), at the C4 position with alkyl, and at the 6 position with phenyl, with 2-(piperidin-4-yloxy)pyrimidine under suitable conditions to prepare a compound of the formula I wherein R¹⁰ is hydrogen and R¹¹ is an alkyl. In an embodiment, R¹¹ is methyl or ethyl.

In another aspect, the invention provides a process for preparing a compound of the formula I wherein R¹⁰ is hydrogen and R¹¹ is aryl which comprises reacting a compound of the formula I wherein R¹⁰ is hydrogen and R¹¹ is halo (e.g., chloro), with pyridazine substituted at the C3 position with halo (e.g., chloro), at the C4 position with aryl, and at the 6 position with phenyl, with 2-(piperidin-4-yloxy)pyrimidine under suitable conditions to prepare a compound of the formula I wherein R¹⁰ is hydrogen and R¹¹ is aryl. In an embodiment, R¹¹ is phenyl.

In another aspect, the invention provides a process for preparing a compound of the formula I wherein R¹⁰ is hydrogen and R¹¹ is an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, more particularly pyridinyl which comprises reacting a compound of the formula I wherein R¹¹ is halo, in particular chloro, and R¹⁰ is hydrogen, with a boronic acid substituted with an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, more particularly pyridinyl, under suitable conditions to prepare a compound of the formula I wherein R¹⁰ is hydrogen and R¹¹ is an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, more particularly pyridinyl.

In an embodiment, the invention provides a process for preparing a compound of the formula I wherein R¹⁰ is hydrogen and R¹¹ is pyridinyl which comprises reacting a compound of the formula I wherein R¹¹ is halo, in particular chloro, and R¹⁰ is hydrogen, with a pyridinyl boronic acid under suitable conditions to prepare a compound of the formula I wherein R¹⁰ is hydrogen and is pyridinyl.

Another aspect, the invention provides a process for preparing a compound of the formula I wherein R¹⁰ is hydrogen and R¹¹ is an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl; or tetrazolyl, more particularly pyridinyl which comprises reacting a pyridazine substituted at the C3 position with halo, at the C4 position with an unsaturated 5 to 6 membered heteromonocyclyl group containing 1 to 4 nitrogen atoms; in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl; triazolyl, or tetrazolyl, more particularly pyridinyl, and at the 6 position with phenyl, with 2-(piperidin-4-yloxy)pyrimidine under suitable conditions to prepare a compound of the formula I wherein R¹⁰ is hydrogen and R¹¹ is an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, more particularly pyridinyl.

In an embodiment, the invention provides a process for preparing a compound of the formula I wherein R¹⁰ is hydrogen and R¹¹ is pyridinyl which comprises reacting a pyridazine substituted at the C3 position with halo, at the C4 position with pyridinyl, and at the 6 position with phenyl, with 2-(piperidin-4-yloxy)pyrimidine under suitable conditions to prepare a compound of the formula I wherein R¹⁰ is hydrogen and R¹¹ is pyridinyl.

In another aspect, the invention provides a process for preparing a compound of the formula I wherein R¹⁰ is hydrogen and R¹¹ is piperidinyl or substituted piperidinyl which comprises reacting a compound of the formula I wherein R¹¹ is halo, in particular chloro, and R¹⁰ is hydrogen with piperazinyl or substituted piperazinyl under suitable conditions to prepare a compound of the formula I wherein R¹⁰ is hydrogen and R¹¹ is piperidinyl or substituted piperidinyl.

In a particular aspect, the invention provides a method for preparing a compound of the Formula I wherein a substituted 6-phenylpyridazine is reacted with 2-(piperazin-1yl)pyridmidine to produce a compound of the Formula I wherein R¹⁰ and R¹¹ are hydrogen. A compound of the formula I wherein R¹⁰ and R¹¹ are hydrogen can be reacted under suitable conditions and with suitable reagents to introduce the radicals R¹⁰ and R¹¹ as define herein.

In embodiments, a process is provided for preparing an acid addition salt of a compound of the formula I wherein R¹⁰ is hydrogen, hydroxyl, alkyl, alkoxy, alkenyl, alkynyl, alkylene, aryl, sulfonyl, sulfinyl, sulfenyl, amino, imino, azido, thiol, thioalkyl, thioalkoxy, nitro, cyano, halo ═O, ═S, carboxyl, carbonyl, carbamoyl, carboxamide, or phosphonate, and R¹¹ is alkyl, alkoxy, alkenyl, aryl, or heteroaryl, more particularly alkyl or heteroaryl, more particularly lower alkyl, which comprises reacting a pyridazine substituted at the C3 position with halo (e.g., chloro), at the C4 position with alkyl, alkoxy, alkenyl, aryl, or heteroaryl, more particularly alkyl or heteroaryl, unsubstituted at C5 or substituted with alkyl, alkoxy, alkenyl, alkynyl, alkylene, aryl, sulfonyl, sulfinyl, sulfenyl, amino, imino, azido, thiol, thioalkyl, thioalkoxy, nitro, cyano, halo ═O, ═S, carboxyl, carbonyl, carbamoyl, carboxamide, or phosphonate, and at the 6 position with phenyl, with 2-(piperidin-4-yloxy)pyrimidine under suitable conditions to prepare a compound of the formula I wherein R¹⁰ is hydrogen and R¹¹ is an alkyl, and reacting with an organic acid in an aqueous solvent medium or in a suitable organic solvent such as methanol, ethanol, or isopropanol. In an embodiment, R¹¹ is methyl, ethyl, or phenyl.

In particular embodiments, a process is provided for preparing an acid addition salt of a compound of the formula I wherein R¹⁰ is hydrogen and R¹¹ is alkyl, alkoxy, alkenyl, aryl, or heteroaryl, more particularly alkyl or heteroaryl, more particularly lower alkyl, which comprises reacting a pyridazine substituted at the C3 position with halo (e.g., chloro), at the C4 position with alkyl, alkoxy, alkenyl, aryl, or heteroaryl, more particularly alkyl or heteroaryl, and at the 6 position with phenyl, with 2-(piperidin-4-yloxy)pyrimidine under suitable conditions to prepare a compound of the formula I wherein R¹⁰ is hydrogen and R¹¹ is an alkyl, and reacting with an inorganic or organic acid, preferably an inorganic acid, in an aqueous solvent medium or in a suitable organic solvent such as methanol, ethanol, or isopropanol. In an embodiment, R¹¹ is methyl, ethyl, or phenyl.

In another particular embodiment, the invention provides a process for preparing an acid addition salt of a compound of the formula I wherein R¹⁰ is hydrogen and R¹¹ is alkyl, alkoxy, alkenyl, aryl, or heteroaryl, more particularly alkyl or heteroaryl comprising reacting a compound of the formula II

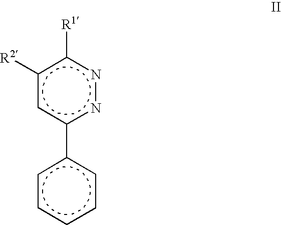

wherein R^{1′} is halo, in particular chloro or bromo, more particularly chloro and R^{2′} is alkyl, alkoxy, alkenyl, aryl, or heteroaryl, more particularly alkyl or heteroaryl with 2-(piperazin-1-yl)pyrimidine under suitable conditions, in particular under reflux conditions to produce a compound of the formula I wherein R¹⁰ is hydrogen and R¹¹ is alkyl, alkoxy, alkenyl, aryl, or heteroaryl, more particularly alkyl or heteroaryl, and reacting with an inorganic acid or organic acid, preferably inorganic acid, in an aqueous solvent medium or in a suitable organic solvent such as methanol, ethanol, or isopropanol. In an embodiment, R¹¹ is C₁-C₆ alkyl. In a particular embodiment, R¹¹ is methyl or ethyl.

In a particular embodiment, a process suitable for large scale manufacture of an acid addition salt of a compound of the formula I wherein R¹⁰ is hydrogen and R¹¹ is alkyl, alkoxy, alkenyl, aryl, or heteroaryl, more particularly alkyl or heteroaryl, is provided comprising:
(a) subjecting a compound of the formula II wherein R^{1′} is ═O and R^{2′} is alkyl, alkoxy, alkenyl, aryl, or heteroaryl, more particularly alkyl or heteroaryl, more particularly lower alkyl, to halogenation conditions to produce a halo-pyridazine compound of the formula II wherein R^{1′} is halo and R^{2′} is alkyl, alkoxy, alkenyl, aryl, or heteroaryl, more particularly alkyl or heteroaryl, more particularly lower alkyl;
(b) reacting the halo-pyridazine compound with a 2-(piperazin-1-yl)pyrimidine under suitable conditions, in particular under reflux conditions, to produce a compound of the formula I wherein R¹⁰ is hydrogen and R¹¹ is alkyl, alkoxy, alkenyl, aryl, or heteroaryl, more particularly alkyl or heteroaryl; and,
(c) reacting with an organic acid in an aqueous solvent medium or in a suitable organic solvent such as methanol, ethanol, or isopropanol.

A compound of the formula II wherein R^{1′} is ═O and R^{2′} is alkyl, alkoxy, alkenyl, aryl, or heteroaryl, more particularly alkyl or heteroaryl, more particularly lower alkyl, may be obtained by (a) cyclizing under suitable conditions a 4-oxo-4-phenylbutanoic acid substituted at the C2 position with alkyl, alkoxy, alkenyl, aryl, or heteroaryl, more particularly alkyl or heteroaryl, most particularly lower alkyl, under suitable conditions to produce 4,5-dihydro-6-phenylpyridazin-3(2H)-one substituted at the C4 position with alkyl, alkoxy, alkenyl, aryl, or heteroaryl, more particularly alkyl or heteroaryl, most particularly lower alkyl; and (b) dehydrogenating the resulting 4,5-dihydro-6-phenylpyridazin-3(2H)-one compound to produce a 6-phenylpyridazin-3(2H)-one substituted at the C4 position with alkyl, alkoxy, alkenyl, aryl, or heteroaryl, more particularly alkyl or heteroaryl, most particularly lower alkyl.

In more particular embodiments of the processes of the invention, R¹¹ is alkyl, preferably C₁-C₆ alkyl, more preferably methyl or ethyl, most preferably methyl. Crystal acid addition salts of a compound of the formula I may be formed by for example, dissolving a hydrochloride or hydrobromide compound of the formula I in a solvent (e.g. methanol), and evaporating the solvent. The crystals may also be prepared by diffusion using standard methods.

In embodiments of the processes of the invention an acid addition salt of a compound of the formula I is produced in a yield of greater than about 80%, 85%, 90%, 92%, 93%, 94%, 95%, 96%, 97%, 98% or 99%.

In a preferred embodiment, the compound is 2-(4-(4-methyl-6-phenylpyridazin-3-yl)piperazin-1-yl)pyrimidine dihydrochloride salt prepared by a production scheme, in particular a GMP production scheme, as shown in FIG. 1.

### Formulations

The invention provides a formulation, dosage form, or pharmaceutical composition, in particular an improved formulation, dosage form, or pharmaceutical composition, comprising a pharmaceutically acceptable acid addition salt of a compound of the formula I, preferably a chloride or bromide salt. A formulation, dosage form, or composition of the invention may be characterized in that the salt of the compound of the formula I has a rate of dissolution of greater than 70, 80, 90 or 100 mg/l per 5 minutes. A formulation, dosage form, or composition may also be characterized by a terminal solubility in the saturation concentration which is maintained for at least one, two or three hours. This formulation, dosage form, or composition can permit the active ingredient to be absorbed rapidly and to a higher degree resulting in improved bioavailability. A formulation, dosage form or composition may also be characterized as having an aqueous solubility at 37° C. of about 100 to 400 mg/ml, about 100 to 350 mg/ml, about 150 to 350 mg/ml, about 200 to 350 mg/ml, or about 300 to 350 mg/ml.

In an aspect of the invention, a solid form pharmaceutical composition is provided (e.g. tablets, capsules, powdered or pulverized form) comprising a crystalline or amorphous salt according to the invention, in particular a chloride or bromide salt of a compound of the formula I. Therefore, a particular composition of the invention is a solid form composition wherein the active ingredient i.e. salt of the invention is in crystalline form. For example, the composition can be in the form of a tablet, capsule, or powder. A particularly preferred solid form composition of the invention having enhanced stability properties comprises a crystalline hydrochloride salt, in particular a di-hydrochloride salt of a compound of the formula I.

In a particular aspect a composition or formulation is provided comprising a non-crystalline salt according to the invention. This formulation may contain an active substance in the form of a dispersion of solids with pharmaceutically acceptable carrier, excipient, or vehicle in a ratio of 1:0.2 to 1:20, in particular 1:0.5 to 1:10 or 1:1 to 1:5.

An aspect of the invention relates to a sterile dosage form of a composition or formulation according to the invention. A composition or formulation may comprise a unit dosage form which comprises all the components of the composition or formulation. In the alternative, the invention encompasses a dosage form kit wherein the acid addition salts of compounds of the formula I and the remaining components of the composition are provided in separate containers and the salts and components are combined prior to administration.

The invention provides pharmaceutical compositions formulated from an acid addition salt of a compound of the formula I (e.g. a chloride or bromide salt preferably a crystalline hydrochloride or hydrobromide salt), a combination of salts of a compound of the formula I, or a combination of a compound of the formula I and an acid addition salt of a compound of the formula I. The compositions include a salt of a compound of the formula I, or include a form of compound of the formula I prepared from a disclosed salt, such as tablets, capsules including a soft gel capsule, or a powdered or pulverized form of the acid addition salt (e.g., halide salt) or other parenteral, transdermal, intranasal or oral administration forms known to the art.

Salts of compounds of the formula I enable the use of a substantially pure active ingredient in pharmaceutical compositions. In aspects of the invention, the salts can have a purity of at least 95%, and preferably at least 97% by weight (e.g. at least 99% to 99.5% by weight). Impurities can include by-products of synthesis or degradation.

A composition of the invention includes one or more pharmaceutical carriers, and optionally one or more bioactive agents. For example, compositions formulated from a halide salt of a compound of the formula I may include: (a) a tablet including a halide salt of a compound of the formula I, a pharmaceutical carrier and may also include an absorption enhancer, (b) a capsule containing a crystalline, amorphous or glassy powder, microspheres, or pellets made from a halide salt (e.g. hydrochloride salt) of a compound of the formula I salt, (c) a soft gel capsule made from a halide salt of a compound of the formula I, (d) an aqueous solution of a halide salt of a compound of the formula I, wherein the dissolved compound is no longer crystals, and may for example, no longer be associated with either the hydrogen or the halide (e.g. chloride or bromide), and (e) other parenteral, transdermal, intranasal or oral administration forms known to those skilled in the art. A compound of the foimula I free base derived from an acid addition salt (e.g. halide salt) disclosed herein is also useful in certain methods of treatment of the invention.

Routes of administration include, without limitation, oral, pulmonary, topical, body cavity (e.g., nasal eye, buccal), transdermal, and parenteral (e.g. intravenous, intramuscular, and subcutaneous routes). Therefore, formulations, dosage forms, and compositions of this invention may be adapted for administration to a subject in a number of ways including but not limited to parenteral (including subcutaneous, intravenous, and intramuscular), oral, mucosal (including buccal, sublingual, and rectal), topical, transdermal and the like.

Formulations include solids (tablets, soft or hard gelatin capsules), semi-solids (gels, creams), or liquids (solutions, colloids, or emulsions), preferably solids. Colloidal carrier systems include microcapsules, emulsions, microspheres, multi-lamellar vesicles, nanocapsules, uni-lamellar vesicles, nanoparticles, microemulsions, and low-density lipoproteins. Formulation systems for parenteral administration include lipid emulsions, liposomes, mixed micellar systems, biodegradable fibers, and fibrin-gels, and biodegradable polymers for implantation. Formulation systems for pulmonary administration include metered dose inhalers, powder inhalers, solutions for inhalation, and liposomes. A composition can be formulated for sustained release (multiple unit disintegrating particles or beads, single unit non-disintegrating system), controlled (oral osmotic pump), and bioadhesives or liposomes. Controlled release formulations include those, which release intermittently, and those that release continuously.

In aspects of the invention an immediate release dosage form is provided comprising an acid addition salt of a compound of the formula I and a drug matrix to effect rapid release of the compound. The invention also provides a sustained release dosage form comprising an acid addition salt of a compound of the formula I and a component for slowing disintegration or dissolution of the compound.

The salts of compounds of the formula I of the invention may be converted into pharmaceutical compositions using customary methods. For example, a hydrochloride or hydrobromide salt of a compound of the formula I, in particular a crystalline hydrochloride or hydrobromide salt of a compound of the formula I, may be mixed into a selected pharmaceutically acceptable carrier, excipient, or diluent as described herein.

Pharmaceutical compositions of the present invention or fractions thereof comprise suitable pharmaceutically acceptable carriers, excipients, and vehicles selected based on the intended form of administration, and consistent with conventional pharmaceutical practices. Suitable pharmaceutical carriers, excipients, and vehicles are described in the standard text Remington: The Science and Practice of Pharmacy. (21st Edition, Popovich, N (eds), Advanced Concepts Institute, University of the Sciences in Philadelphia, Philadelphia, Pa. 2005). Pharmaceutical carriers include, for example, inorganics such as calcium phosphate and titanium dioxide; carbohydrates such as d(+)-lactose monohydrate and cyclodextrin; surfactants such as sodium lauryl sulfate and poloxamers; polymers such as starch, ethyl cellulose, hydrogels, and polyacrylic acids; lipids such as polylactides, stearic acid, glycerides, and phospholipids; or amino acids and peptides such as leucine and low density lipoprotein.

A composition is generally formulated so that it remains active at physiologic pH. A composition may be formulated in the pH range 4 to 9, in particular 4 to 7, more particularly 5 to 7.

In an embodiment of the invention a composition is provided which is an oral dosage form comprising a salt of a compound of the formula I (in particular a halide salt, more particularly a hydrochloride salt, most particularly a crystalline hydrochloride salt) and a non-hygroscopic, inert and preferably anhydrous excipient (e.g. lactose or mannitol). In another embodiment, a composition is provided which is a soft gelatin capsule comprising a salt of a compound of the formula I (in particular a halide salt, more particularly a hydrochloride salt most particularly a crystalline hydrochloride salt) and at least one hydrophilic vehicle (e.g. glycerin or propylene glycol) and at least one lipophilic vehicle (e.g. PEG 400).

Compositions may also include absorption enhancers, particle coatings (e.g. enteric coatings), lubricants, targeting agents, and any other agents known to one skilled in the art. A composition may contain from about 0.1 to 90% by weight (such as about 0.1 to 20% or about 0.5 to 10%) of the active ingredient.

A composition of the invention may be sterilized by, for example, filtration through a bacteria retaining filter, addition of sterilizing agents to the composition, irradiation of the composition, or heating the composition. Alternatively, the compositions, of the present invention may be provided as sterile solid preparations e.g. lyophilized powder, which are readily dissolved in sterile solvent immediately prior to use.

After pharmaceutical compositions have been prepared, they can be placed in an appropriate container and labeled for treatment of an indicated condition. For administration of a composition of the invention, such labeling would include amount, frequency, and method of administration.

According to the invention, a kit is provided. In an aspect, the kit comprises salts of compounds of the formula I or formulations, dosage forms or compositions of the invention in kit form. The kit can be a package which houses a container which contains salts of compounds of the formula I, or formulations, dosage forms or compositions of the invention and also houses instructions for administering the compounds, formulations, dosage forms or compositions to a subject. The invention further relates to a commercial package comprising salts of compounds of the formula I, formulations, dosage forms or compositions of the invention together with instructions for simultaneous, separate or sequential use. In particular a label may include amount, frequency, and method of administration.

The invention also provides a pharmaceutical pack or kit comprising one or more containers filled with one or more of the ingredients of a formulation, dosage form or composition of the invention to provide a beneficial effect. Associated with such container(s) can be various written materials such as instructions for use, or a notice in the form prescribed by a governmental agency regulating the labeling, manufacture, use or sale of pharmaceuticals or biological products, which notice reflects approval by the agency of manufacture, use, or sale for human administration.

The invention also relates to articles of manufacture and kits containing materials useful for treating a disease disclosed herein. An article of manufacture may comprise a container with a label. Examples of suitable containers include bottles, vials, and test tubes which may be formed from a variety of materials including glass and plastic. A container holds salts of compounds of the formula I or formulations, dosage forms or compositions of the invention which are effective for treating a disease disclosed herein. The label on the container indicates that the salts of compounds of the formula I or formulations, dosage forms or compositions of the invention are used for treating a disease disclosed herein and may also indicate directions for use. In aspects of the invention, a formulation, dosage form or composition in a container may comprise any of the formulations, dosage forms or compositions disclosed herein.

The invention also contemplates kits comprising one or more salts of compounds of the formula I. In aspects of the invention, a kit of the invention comprises a container described herein. In particular aspects, a kit of the invention comprises a container described herein and a second container comprising a buffer. A kit may additionally include other materials desirable from a commercial and user standpoint, including, without limitation, buffers, diluents, filters, needles, syringes, and package inserts with instructions for performing any methods disclosed herein (e.g., methods for treating a disease disclosed herein).

In aspects of the invention, the kits may be useful for any of the methods disclosed herein, including, without limitation treating a subject suffering from Alzheimer's disease. Kits of the invention may contain instructions for practicing any of the methods described herein.

The invention provides a method for treating a disease disclosed herein in a subject comprising administering an effective amount of a salt, formulation, dosage form or composition according to the invention. The invention also relates to the use of a salt, formulation, dosage form or composition according to the invention in the preparation of a medicament for treating a disease disclosed herein. Salts, formulations, dosage forms and compositions of the invention may be administered therapeutically or prophylactically to treat a disease disclosed herein, in particular a neuroinflammatory disease.

The percentage of active ingredient in each formulation, dosage form, or composition and the therapeutically effective amount of the active ingredient used to practice the present invention for treatment of the disclosed diseases and conditions depend upon the manner of administration, the age and the body weight of the subject and the condition of the subject to be treated, and ultimately will be decided by the attending physician or veterinarian. Examples of suitable dose ranges include about 0.01 to 3000 mg/kg, 0.01 to 2000 mg/kg, 0.5 to 2000 mg/kg, about 0.5 to 1000 mg/kg, 0.1 to 1000 mg/kg, 0.1 to 500 mg/kg, 0.1 to 400 mg/kg, 0.1 to 300 mg/kg, 0.1 to 200 mg/kg, 0.1 to 100 mg/kg, 0.1 to 50 mg/kg, 0.1 to 20 mg/kg, 0.1 to 10 mg/kg, 0.1 to 6 mg/kg, 0.1 to 5 mg/kg, 0.1 to 3 mg/kg, 0.1 to 2 mg/kg, 0.1 to 1 mg/kg, 1 to 1000 mg/kg, 1 to 500 mg/kg, 1 to 400 mg/kg, 1 to 300 mg/kg, 1 to 200 mg/kg, 1 to 100 mg/kg, 1 to 50 mg/kg, 1 to 20 mg/kg, 1 to 10 mg/kg, 1 to 6 mg/kg, 1 to 5 mg/kg, or 1 to 3 mg/kg, or 1 to 2.5 mg/kg, or less than or about 10 mg/kg, 5 mg/kg, 2.5 mg/kg, 1 mg/kg, or 0.5 mg/kg twice daily or less.

In aspects of the invention, a formulation or dosage form suitable for once, twice a day, or three times a day or more administration is provided comprising one or more salts of compounds of the formula I present in an amount sufficient to provide the required concentration or dose of the compounds to an environment of use to treat a disease disclosed herein, in particular a neuroinflammatory disease. In an aspect, the environment of use is the brain and/or plasma.

A subject may be treated with a salt of a compound of the formula I or formulation, composition or unit dosage thereof on substantially any desired schedule. They may be administered one or more times per day, in particular 1 or 2 times per day, once per week, once a month or continuously. However, a subject may be treated less frequently, such as every other day or once a week, or more frequently. A compound, formulation, dosage unit or composition may be administered to a subject for about or at least about 24 hours, 2 days, 3 days, 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, 2 weeks to 24 months, or for more than 24 months, periodically or continuously.

In an aspect, a formulation, composition or dosage form is provided that is suitable for once, twice, or three times a day administration, preferably twice a day administration comprising one or more salt of a compound of the formula present in an amount sufficient to provide the required dose of the compound. In an aspect, the required dose of a salt of a compound of the formula I administered once twice, three times or more daily is about 0.1 to 1000 mg/kg, 0.1 to 500 mg/kg, 0.1 to 400 mg/kg, 0.1 to 300 mg/kg, 0.1 to 200 mg/kg, 0.1 to 100 mg/kg, 0.1 to 75 mg/kg, 0.1 to 50 mg/kg, 0.1 to 25 mg/kg, 0.1 to 20 mg/kg, 0.1 to 15 mg/kg, 0.1 to 10 mg/kg, 0.1 to 9 mg/kg, 0.1 to 8 mg/kg, 0.1 to 7 mg/kg, 0.1 to 6 mg/kg, 0.1 to 5 mg/kg, 0.1 to 4 mg/kg, 0.1 to 3 mg/kg, 0.1 to 2 mg/kg, or 0.1 to 1 mg/kg.

Embodiments of the invention relate to a dosage form comprising one or more addition salt of a compound of the formula I that provides peak plasma concentrations of the compound, Cₘₐₓ, of between about 0.001 to 2 mg/ml, 0.001 to 1 mg/ml, 0.002 to 2 mg/ml, 0.005 to 2 mg/ml, 0.01 to 2 mg/ml, 0.05 to 2 mg/ml, 0.1 to 2 mg/ml, 0.001 to 0.5 mg/ml, 0.002 to 1 mg/ml, 0.005 to 1 mg/ml, 0.01 to 1 mg/ml, 0.05 to 1 mg/ml, or 0.1 to 1 mg/ml.

In further aspects, the invention provides a formulation or dosage form comprising one or more addition salt of a compound of the formula I that provides an elimination t_{1/2} of 0.1 to 20 hours, 0.1 to 10 hours, 0.1 to 6 hours, 0.2 to 20 hours, 0.2 to 10 hours, 0.2 to 6 hours, 0.25 to 10 hours, 0.25 to 6 hours, 0.5 to 20 hours, 0.5 to 15 hours, 0.5 to 10 hours, 0.5 to 6 hours, 1 to 20 hours, 1 to 15 hours, 1 to 10 hours, or 1 to 6 hours.

Further aspects of the invention relate to a formulation or dosage form comprising one or more acid addition salt of a compound of the formula I that provides an AUC for plasma of about 3 to 2000 ng·h/ml, 3 to 3000 ng·h/ml, 3 to 4000 ng·h/ml, 2 to 2000 ng·h/ml, 2 to 3000 ng·h/ml, 2 to 4000 ng·h/ml, 1 to 2000 ng·h/ml, 1 to 3000 ng·h/ml, 1 to 4000 ng·h/ml, 1, and in particular 3 to 3000 ng·h/ml

Salts, formulations, dosage forms and compositions of the invention may also be co-administered with one or more additional therapeutic methods or agents. Examples of such agents include, without limitation, inhibitors of beta-sheet aggregation/fibrillogenesis/ADDL formation (e.g. Alzhemed), NMDA antagonists (e.g. memantine), anti-oxidants (e.g. Vitamin E), hormones (e.g. estrogens), nutrients and food supplements (e.g. Gingko biloba), statins and other cholesterol lowering drugs (e.g. Lovastatin and Simvastatin), secretase inhibitors, acetylcholinesterase inhibitors (e.g. donezepil), muscarinic agonists (e.g. AF102B (Cevimeline, EVOXAC), AF150(S), and AF267B), anti-psychotics (e.g. haloperidol, clozapine, olanzapine), anti-depressants including tricyclics and serotonin reuptake inhibitors (e.g. Sertraline and Citalopram Hbr), immunotherapeutics and antibodies to Aβ (e.g. ELAN AN-1792), vaccines, inhibitors of kinases (CDK5, GSK3α, GSK3β) that phosphorylate TAU protein (e.g. Lithium chloride), inhibitors of kinases that modulate Aβ production (GSK3α, GSK3β, Rho/ROCK kinases) (e.g. lithium Chloride and Ibuprofen), drugs that upregulate neprilysin (an enzyme which degrades Aβ); drugs that upregulate insulin degrading enzyme (an enzyme which degrades Aβ), agents that are used for the treatment of complications resulting from or associated with a disease, or general medications that treat or prevent side effects. The present invention also includes methods of using the formulations, dosage forms or compositions of the invention in combination treatments with one of more additional treatments including without limitation gene therapy and/or drug based approaches to up-regulate neprilysin (an enzyme which degrades Aβ), gene therapy and/or drug based approaches to upregulate insulin degrading enzyme (an enzyme which degrades Aβ), or stem cell and other cell-based therapies. The salt of compounds of the invention may be administered concurrently, separately, or sequentially with other therapeutic agents or therapies.

The invention will be described in greater detail by way of specific examples. The following examples are offered for illustrative purposes, and are not intended to limit the invention in any manner.

### EXAMPLES

### Example 1

### Preparation of 4-methyl-6-phenyl-3(4-pyrimidin-2-ylpiperazin-1-yl)pyridazine (MW01-2-151SRM)

4-methyl-6-phenyl-3-(4-pyrimidin-2-yl)piperazin-1-yl)pyridazine (MW01-2-151 SRM) can be prepared by the synthetic scheme depicted in FIG. 1 which was carried out as described in detail herein.

### 4,5-dihydro-4-methyl-6-phenylpyridazin-3(2H)-one (2)

A 250 mL three-neck round bottom flask fit with a temperature probe and condenser is charged with 7.7 g (40 mmole) of 2-methyl-4-oxo-4-phenylbutanoic acid 1 and 20 ml of ethanol (95%). The suspension is cooled to below 10° C. and 2.2 ml (42 mmole, 1.05 equiv.) of hydrazine monohydrate in 10 mL of ethanol is added dropwise at a rate that maintains the solution temperature at below 20° C. Upon addition, the suspension changes to a pale yellow solution. After addition, the reaction mixture is heated to reflux and stirred for 2 h, and after 20 minutes of heating, a solid is seen in the mixture; Once the reaction is completed, the flask is removed from the oil bath and cooled to ambient temperature. Upon cooling, white crystals form in the flask, which are collected by filtration. The solid is washed first with 30 mL of 2N NaHCO₃, followed by 60 mL Milli-Q water three times, and dried over a medium frit, sintered glass funnel in vacuo to give the desired product 2 in 96.1% yield. [See Hansen, K B et al. Organk process research & development, 2005, 9, 634-639; Nelson, D A. US 20050137397A1. Coudert, P et al. Journal of Heterocyclic Chemistry, 1988, 25(3), 799-802.]

### 4-methyl-6-phenylpyridiazin-3(2H)-one (3)

7.0 g (35 mmole) of 2 is placed in a 250 ml single-necked round bottom flask followed by 3.0 mL of acetonitrile. The mixture is stirred to allow 2 to dissolve. 11.3 g (84 mmole, 2.4 equiv.) of anhydrous copper (II) chloride is added to the solution to give a green-yellow suspension. A reflux condenser is connected to the flask and a dry tube filled with anhydrous CaCl₂ is fitted to the top of the condenser. To control the HCl gas that forms during the course of the reaction, a NaOH solution is used to absorb the HCl that escapes from the dry tube. The reaction mixture is heated to reflux, and the color of the reaction suspension changes to dark green upon heating. When the reaction is complete (after refluxing for 2 h), the flask is removed from the oil bath and cooled to ambient temperature. The reaction is cooled in an ice-water bath and 150 mL of ice-water is added to quench the reaction. The mixture is stirred vigorously for 10 minutes to give a gray precipitate and blue liquid containing copper (I) chloride. The precipitate is collected by filtration (pH of the filtrate is 0-1) and washed with 100 mL of 1N HCl solution, then 100 mL of water 5 times. To remove remaining copper by-products that are trapped in the solid, the filter cake is stirred in 150 mL of 1N HCl solution for 0.5 h and filtered. The filter cake is subsequently washed with Milli-Q water until the filtrate is at pH 7 (approximately 7 washes). The solid is dried over a medium frit sintered glass funnel in vacuo to give 3 as a light gray powder in 93.8% yield. [See Eddy, S et al. Synthetic Communications, 2000, 30(1), 1-7 Csende, F et al. Synthesis, 1995, 1240-1242.]

### 3-chloro-4-methyl-6-phenylpyridazine (4)

6.0 g (32 mmole) of 3 is placed in a 250 mL single neck round bottom flask and 30 mL of acetonitrile is added to create a pale yellow slurry. 6.0 ml (64 mmole, 2 equiv.) of phosphorus oxychloride is added changing the slurry to a darker color. The flask is fitted with a reflux condenser and a dry tube filled with anhydrous CaCl₂ is fitted to the top of the condenser. The reaction mixture is heated at reflux and becomes a dark red liquid. After the reaction is completed (2.5 h); the mixture is cooled to ambient temperature and placed in an ice water bath. Ice water (150 mL) is slowly poured into the reaction mixture with stirring to decompose the phosphorus oxychloride into HCl and H₃PO₄, resulting in formation of a pink solid. The solid is collected by filtration and washed three times with 50 mL of Milli-Q water. The solid is transferred to a 250 mL beaker, followed by addition of 100 mL of water to form a suspension. Subsequently, 1N NaOH is added until the aqueous suspension is at pH=8, and the mixture is stirred for 5 minutes to remove all trace of starting material contaminants. The solid is filtered and washed 3 times with 100 mL of water to wash out the excess base. The solid is dried over a medium frit sintered glass funnel in vacuo to provide 4 as a light pink powder in 96% yield. [See Contreras, J M et al. Journal of Medicinal Chemistry, 2001, 44(17), 2707-2718; Nelson, D A. US 20050137397A1.]

### 2-(4-(4-methyl-6-phenylpyridazin-3-yl)piperazin-1-yl)pyrimidine (5)

7.5 g (36.6 mmole) of 4 is placed in a 250 mL single neck round bottom flask and suspended in 125 mL of water. 60.17 g (366.0 mmole, 10 equiv.) of 2-(piperazine-1-yl)pyrimidine is added and the flask fit with a condenser. The reaction mixture is heated at reflux with rapid stirring for 60 h, with continuous amine addition possible to boost reaction rates. When complete, the reaction mixture is cooled to ambient temperature and two layers are observed in the flask consisting of an orange aqueous layer and a brown oil that settles to the bottom of the flask. The water is decanted off, leaving the oil, which is the product 5. The oil is then dissolved in minimal volume of isopropanol and heated to reflux. After 10 minutes of reflux, the solution is cooled to ambient temperature, and cooled to 0° C. to induce crystallization. Pale yellow crystals are filtered from isopropanol and rinsed with minimal cold ether to provide 5. Recovery of the crystals is 50%, but may be increased by recursive crystallization of compound. [Contreras, J M et al. Journal of Medicinal Chemistry, 1999, 42(4), 730-741. Chayer, S et al. Tetrahedron Letters, 1998, 39, 841-844.]

### 2-(4-(4-methyl-6-phenylpyridazin-3-yl)piperazin-1-yl)pyrimidine dihydrochloride salt (6)

6.3 g (19.0 mmol) of 5 is suspended in 50 mL of anhydrous isopropanol, heated to 70° C. with stirring until the solid is dissolved into a dark red solution. 2.5 eq (4.0 mL) of concentrated HCl is added at once to the solution, causing an immediate change to a yellow suspension. The suspension is stirred at 70° C. for 10 min, cooled to ambient temperature and cooled on ice for 0.5 h. The precipitate is collected by filtration and washed once with 30 mL cold isopropanol to provide the product 6 as a yellow powder in 93.3% yield. Elemental analysis shows that 6 is the monohydrate of the dihydrochloride salt of 5. [Wermuth C G, Stahl PH. Selected Procedures for the Preparation of Pharmaceutically Acceptable Salts, in Stahl P H., Wermuth C G. (Ed.) Handbook of Pharmaceutical Salts, Wiley-VCH, p 249-264.]

### Example 2

A lead compound, MW01-5-188WH (compound 1) was subjected to molecular property driven medicinal chemistry refinement to improve aqueous solubility without increasing molecular weight while retaining its in vivo functions (e.g. attenuation of human amyloid-beta (Aβ-induced up-regulation of glia proinflammatory cytokine production, synaptic dysfunction, and hippocampus-dependent behavioral deficits).

Position β was varied first (compounds 2-6) to explore improvement in logS with retention of activity while gaining insight into the relationship between structure and activity (SAR). All compounds in this group have an improved computed logS compared to compound 1. Compounds 2 and 5 retained selective suppression of IL-1α production compared to NO production, compounds 4 and 6 lost selectivity, and compound 3 was less active (Table 1). When R4=H, the compound is inactive. As is evident from inspection of Table 1, compound 5 retains activity, has a decreased MW, and has an improved logS and logP. With R4=CH₃, variation at R3 was then pursued (compounds 7-11). Most changes resulted in a loss of activity (compounds 7, 9, 10 and 11). Compound 8 was the only analog that was comparable to compound 5 in activity and molecular properties.

The results with compounds 5 and 8 demonstrate that molecular property-driven refinement can improve logS, reduce MW, and retain selective activity. Compound 5 was chosen for development and testing as an active pharmaceutical ingredient (API).

Compound 5 was initially synthesized with a Pd-catalyzed reaction from a common 4-chloropyridazine precursor (compound 6). However, this protocol is not readily scalable for production under GMP conditions. To facilitate manufacture of this drug candidate, a validated and cost-effective, non-toxic and high yield protocol was developed which is outlined in Scheme 1 (FIG. 1).

The commercially available acid 12 was cyclized with hydrazine to obtain 13, which was then dehydrogenated to 4-methyl-6-phenylpyridazinone 14. Chlorination with phosphorus oxychloride provided the 3-chloropyridazine 15. Compound 5 was then obtained by amination. The corresponding hydrochloride hydrate 16 was synthesized by treatment with HCl in isopropanol.

Compound 16 is water soluble and stable. Compound 16 demonstrates improvement in aqueous solubility at 37° C. compared to the corresponding hydrochloride hydrate of lead compound 1, with experimental values of 322 mg/ml and 0.04 mg/ml, respectively. In contrast to the differences in experimentally determined aqueous solubility, the lead compound 1 and drug candidate 16 possess similar experimentally determined logP values (2.3 and 2.7), as measured by octanol/water partition coefficient. Compound 16 has a melting point over 215° C., a temperature at which it decomposes, compared to a melting point of approximately 116.1° C. for the base compound 5. When compound 16 was tested for chemical stability in water, dilute acid, or dilute base at 37° C. over one week, there was less than 10% loss of material. These data provide experimental confirmation that the molecular properties were improved as a result of refinement, and demonstrate that the API is a stable, water soluble product.

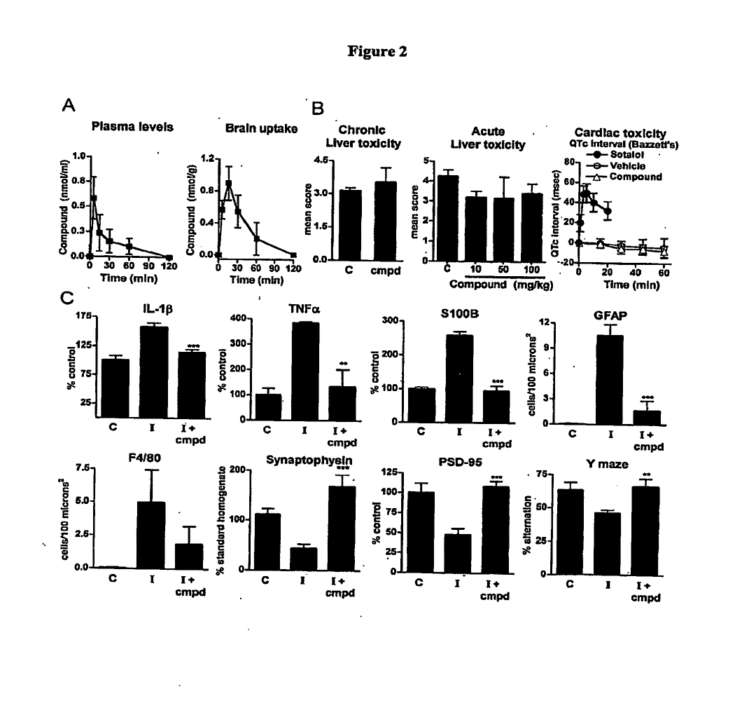

To test for retention of in vivo function after refinement, compound 16 was subjected to the same battery of tests that resulted in the discovery of the lead compound 1. As shown in FIG. 2, the in vivo bioavailability, good safety profile, and in vivo efficacy in an animal model of disease were all retained. FIG. 2A shows that the compound rapidly appears in the plasma and brain after a single oral gavage administration to mice, indicative of good oral bioavailability and brain uptake characteristics. The compound is non-toxic, in that there is no histological liver toxicity after chronic oral administration of a therapeutic dose (FIG. 2B, left) or after acute, escalating-dose, oral administration at concentrations up to 40 times the therapeutic dose (FIG. 2B, center). There is no evidence of in vivo cardiac toxicity, as measured by prolongation of cardiac QTc interval (FIG. 2B, right).

Compound 16 is efficacious in an AD relevant mouse model of human Aβ-induced injury. Daily oral administration of compound at a low dose (2.5 mg/kg) once daily for 2 weeks, beginning 21 days after initiation of the human Aβ injury, effectively suppressed the disease outcomes in hippocampus harvested at day 60 after the start of injury (FIG. 2C). The Aβ-induced up-regulation of the proinflammatory cytokines interleukin-1β (IL-1β, tumor necrosis factor alpha (TNFα), and S100B were suppressed.

The number of activated GFAP-positive astrocytes and F4/80-positive microglia were reduced. The loss of the synaptic proteins synaptophysin and PSD-95 was attenuated. Deficits in the Y-maze, a hippocampus dependent behavioral task, were ameliorated. These data document that the title compound retains the desired in vivo functions of the lead compound 1.

In summary, a novel, water soluble, disease-modifying new class of therapeutic that has a promising safety and bioavailability profile has been identified. An organic process scheme has also been developed that is amenable to large scale GMP production with good yield. The medicinal chemistry refinement of a lead compound was based on improvement of aqueous solubility with retention of in vivo functions, which was achieved with the development of compound 5 and its hydrochloride hydrate salt, compound 16, as an API. Although refinement was focused on improvement of molecular properties with retention of function, the results also provided insight into SAR for selective suppression of glial activation pathways. Especially interesting was the dependence of selective activity on the structures associated with the R3 amine. For example, there appears to be a strong preference for an aromatic group on the piperazine for full activity, based on the diminution of activity with the presence of a cyclohexyl (compound 10) or methyl (compound 11) group. This suggestion was followed up in unrelated studies by making the corresponding analog containing a phenylpiperazine, compound 22, which was found to be active. Although not an improvement in molecular properties, it confirms that selective inhibition of up-regulated proinflammatory cytokine production in glia can be achieved by one of several structures in the context of a 3-piperazinyl-4-alkyl-6-arylpyridazine scaffold.

### Example 3

### In Vivo Studies

Effect of MW01-2-151SRM (also Referred to Herein as Minozac) in an Animal Model of Alzheimer's Disease (AD) (Aβ Infusion Model)

The efficacy of Minozac in an AD-relevant mouse model of human amyloid-beta (Aβ)-induced injury (infusion of human Aβ peptide) was determined. Briefly, C57BL/6 mice (n=5-10 mice per group) were subjected to either vehicle (Control) or human oligomeric Aβ1-42 intracerebroventricular (ICV) infusion for four weeks. At three weeks after the start of infusion and continuing for two weeks thereafter, a single, daily oral dose of Minozac at a low dose (2.5 mg/kg) was administered by oral gavage. Beginning at day 50 after the start of Aβ ICV infusion, the Y-maze test of spontaneous alternation was used to evaluate hippocampus-dependent spatial learning deficits. At day 60 after the start of Aβ ICV infusion, mice were sacrificed, perfused with buffer containing a protease inhibitor cocktail, and the brain was harvested. The brain was longitudinally bisected and the right half of the brain used for histology. The hippocampus was dissected from the left half of the brain. The levels of IL-1β, TNFα, S100B and synaptophysin were measured in hippocampal extracts by ELISA, and postsynaptic density-95 (SD-95) levels were determined by Western blotting. Immunohistochemical detection of GFAP-positive astrocytes and F4/80 positive microglia was performed on 10 μm sections.

Minozac treatment effectively suppressed the upregulation of the proinflammatory cytokines interleukin-1β (IL-1β), tumor necrosis factor alpha (TNFα), and S100B measured in hippocampal extracts. The number of activated GFAP-positive astrocytes and F4/80-positive microglia were also reduced in hippocampus. The loss of the synaptic marker proteins synaptophysin and PSD-95 was attenuated in hippocampal extracts. Deficits in the Y-maze test, a hippocampus-dependent behavioral task, were ameliorated.

### Effect of Minozac on Systemic Proinflammatory Cytokine Expression

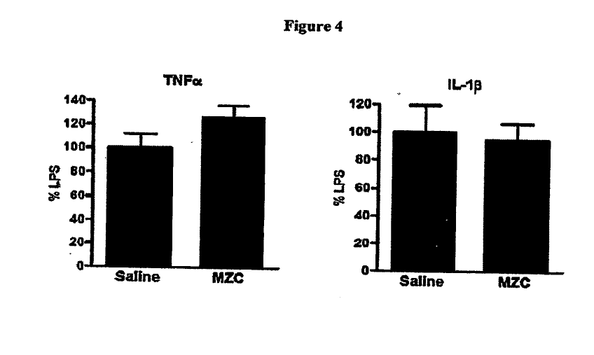

The effect of Minozac on the systemic expression of proinflammatory cytokines was determined. Briefly, saline (0.9% NaCL) or Minozac (15 mg/kg) was administered orally for 14 days. Mice were challenged with LPS at the 15th day. Mice were sacrificed at 6 hours after LPS challenge and blood harvested for serum. The levels of cytokines TNFα and IL-113 in the serum were measured using the Meso-scale Discovery system according to manufacturer's instructions. The data for TNFα and IL-1β are shown in FIG. 4 as percent of the level observed in the saline treated mice. In contrast to the effects of Minozac on the expression of TNFα and IL-1β in the hippocampus at a dose of 2.5 mg/kg in AB ICV mouse model, there is no effect of Minozac on the expression of these same cytokines systemically in an LPS inflammation model.

### Example 4

### Protocol

Plasma samples obtained from dog pharmacokinetic studies were analyzed using reversed phase HPLC with tandem mass spectrometric detection. Briefly, aliquots of internal standard (200 μl of 200 ng/ml) were added to protein precipitation plates (Sirocco protein precipitation plates, Waters Corporation, Milford, Mass., USA), followed by addition of plasma sample to the plate (50 μl). The plate was shaken for 3 minutes, and then a vacuum (maximum of −10 mm Hg) was applied to the plate for 3 minutes. Extract (75 μl) was transferred into a 2 ml 96-well plate, and to each sample was added 1425 μl of Eluent A (95:5:0.1 MilliQ water:acetonitrile:formic acid). 10 μl was injected onto the reverse phase column (ODS C18; 4×2 mm i.d.; Phenomenex, Torrance, USA). The mobile phase was set to 300 μl/min and eluate by mass spectrometry (APT 5000, Applied Biosystems). Calibration standards were processed along with the samples, and the concentration of Minozac [2-(4-(4-methyl-6-phenylpyridazin-3-yl)piperazin-1-yl)pyrimidine dihydrochloride salt] determined using a regression equation. Retention times of Minozac and internal standard were 3.8 and 2.8 minutes, respectively. The internal standard was compound code MW01-8-064WH (chemical name: 4-methyl-3-phenyl-6-((4-pyrimidin-2-yl)piperazin-1-yl)pyridazine).

Plasma samples obtained from mouse pharmacokinetic studies were analyzed by reversed phase HPLC with ultraviolet detection. Briefly, cartridges (Sep-Pak® C18, Waters) were conditioned with 1 ml of acetonitrile (HPLC grade, EMD Biosciences) and equilibrated with 1 ml of water. A structural analog, 6-methyl-4-phenyl-3-(4(pyrimidin-2-yl)piperazin-1-yl)pyridazine (MW01-7-057WH), was used as an internal standard. Acidified samples were loaded onto the cartridge followed by a 1 ml wash with 10% acetonitrile. Minozac was eluted from the cartridge using 80% acetonitrile. The eluate was evaporated to dryness and reconstituted in 0.08% formic acid/water in 80% acetonitrile. Sample was injected onto a Dionex HPLC system (Dionex, Sunnyvale, Calif.) using a Phenomenex (Torrance, Calif.) Luna C18 column (250×2.0 mm; 5 μm) and guard column with a flow rate of 0.2 ml/min. Peak quantification was performed based upon absorption at 254 nm relative to a standard curve obtained by serial dilutions of the compound.

### PK Assessment of Minozac in C57BU6 Mice

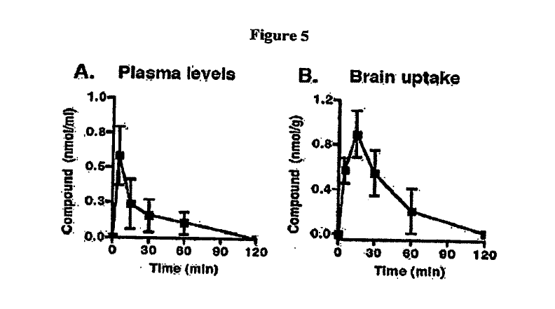

Minozac (2.5 mg/kg) was well absorbed following oral administration by gavage in a 0.5% (w/v) carboxymethylcellulose suspension. Maximum plasma concentration (Cₘₐₓ) was reached at approximately 7.5 minutes with a t_{1/2} of about 15 minutes. The time course of distribution of Minbzac to the brain followed closely the time course of drug in plasma, with Tₘₐₓ in brain tissue (determined in whole brain homogenates) reached at approximately 15 minutes with a t_{1/2} of about 45 minutes (FIG. 5).

### PK Assessment of Minozac in Beagle Dog

The pharmacokinetics of Minozac was determined at 2.5 mg/kg following oral administration by gavage of an aqueous solution. Consistent with observations made in the mouse study, absorption was rapid with a Tₘₐₓ of 0.25 hour. After absorption, Minozac was rapidly eliminated from the body with a half-life of 2.10 hour. Bioavailability of Minozac after oral dosing was 23%.

Intravenous dosing was also carried out at 2.5 mg/kg, but with drug in a 0.9% saline solution. The volume of distribution associated with the terminal phase (Vz) was approximately twice as high as the steady-state volume of distribution (Vss), which suggests a significant distribution phase of the compound. The terminal half-life was 4.49 hour. The apparent terminal half-life after intravenous dosing was over twice that observed after oral dosing. The pharmacokinetic properties are summarized in Table 2.

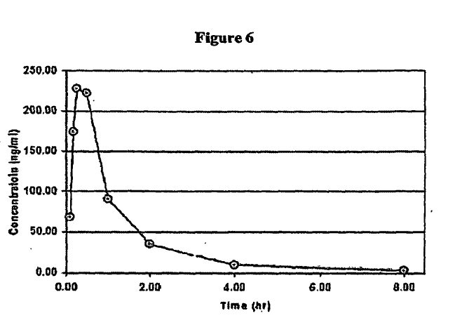

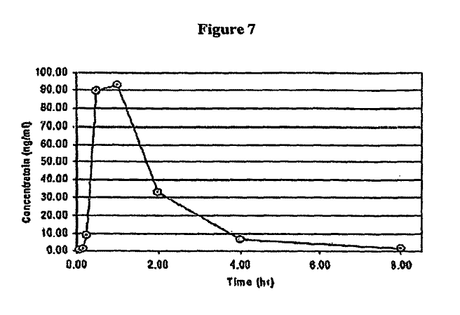

The pharmacokinetics were determined following oral dosing with a neutralized propylene glycol formulation (FIG. 6) and with filled gelatin capsules at a single dose of 2.5 mg/kg (FIG. 7).

A summary of the PK parameters of Minozac after single administration to male dogs by oral gavage with a pH neutral propylene glycol solution (Period 1) or filled gelatin capsule (Period 2) are given in Table 3. The bioavailability after an oral gavage and capsule administration was 21% and 16%, respectively. These data are comparable to the pharmacokinetic data obtained following oral administration by gavage of an aqueous solution of drug.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the methods etc. which are reported therein which might be used in connection with the invention. Nothing herein is to be construed as an admission that the invention is not entitled to antedate such disclosure by virtue of prior invention.

TABLE I
Medicinal chemistry refinement

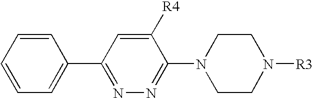

| | R4 | R3 | MW* | log S* | log P* | IL-Iβ^{⊥} | NO^{⊥} |
|---|---|---|---|---|---|---|---|
| 1 | 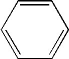 | 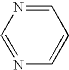 | 394.47 | −5.40 | 3.88 | 2.5 ≐ 1.6 | >25 |
| 2 | 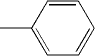 | 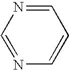 | 408.50 | −5.33 | 3.82 | 5.3 ≐ 0.6 | >25 |
| 3 | 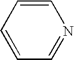 | 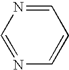 | 395.46 | −4.41 | 2.49 | 25.8 ≐ 7.0 | >25 |
| 4 | 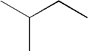 | 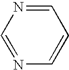 | 374.48 | −4.89 | 3.71 | 6.1 ≐ 2.5 | 22 ≐ 3 |
| 5 | CH₃ | 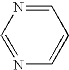 | 332.40 | −4.08 | 2.29 | 8.3 ≐ 5.8 | >25 |
| 6 | Cl | 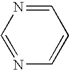 | 352.82 | −4.64 | 2.76 | 9.5 ≐ 4.0 | 19 ≐ 3 |
| 7 | CH₃ | 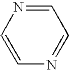 | 332.40 | −1.48 | 2.01 | 46.1 ≐ 23.3 | |
| 8 | CH₃ | 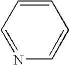 | 331.41 | −2.11 | 2.45 | 7.6 ≐ 2.9 | >25 |
| 9 | CH₃ | 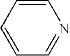 | 331.41 | −2.09 | 2.38 | 17.7 −7.2 | >25 |
| 10 | CH₃ | 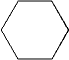 | 336.47 | −2.80 | 3.88 | 31.4 ≐ 4.9 | |
| 11 | CH₃ | CH₃ | 268.36 | −1.22 | 1.83 | 48.9 ≐ 22.2 | |

*Calculated using ACD/Solubility DB 9.03.
logS is intrinsic solubility of neutral form of compounds.
PSA: 1, 2, 4-7 = 58.04; 3 = 70.93; 8, 9 = 45.15; 10, 11 = 32.26.
^{⊥}Concentration (μM) required for 50% inhibition⁸.
IL-1β = interleukin-1β; NO = nitric oxide.

TABLE 2

| Pharmacokinetic | | |
|---|---|---|
| parameters | | Minozac |
| Intravenous dosing | | |
| Dose | mg | 19.4 ± 1.38 |
| AUC∞ | hr * ng/ml | 1457 ± 490 |
| #AUC∞ | hr * ng/ml/mg | 74.4 ± 22.5 |
| t_{1/2} | hr | 4.49 ± 1.12 |
| CI | L/hr | 14.5 ± 5.29 |
| V_{z} | L | 95.1 ± 43.6 |
| Vₛₛ | L | 44.5 ± 10.3 |
| MRT∞ | hr | 3.28 ± 1.20 |
| Oral dosing | | |
| Dose | mg | 18.9 ± 1.15 |
| tₘₐₓ | hr^{$} | 0.25 ± n/a |
| Cₘₐₓ | ng/ml | 300 ± 172 |
| AUC∞ | hr * ng/ml | 314 ± 187 |
| #AUC∞ | hr * ng/ml/mg | 16.9 ± 10.6 |
| t_{1/2} | hr | 2.10 ± 0.755 |
| F | % | 23 |

^{$}median
#dose normalized to 1 mg

TABLE 3

| | Period 1 | Period 2 |
|---|---|---|
| | Gavage | capsule |
| Tₘₐₓ (hr) | 0.17 | 0.50 |
| Cₘₐₓ (ng/ml) | 288 | 447 |
| AUC_{∞} (hr * ng/ml) | 299 | 237 |
| t_{1/2} (hr) | 1.47 | 1.19 |

What is claimed is:

1. A stable and substantially purified synthetic pharmaceutically acceptable acid addition salts of a compound of the formula I: 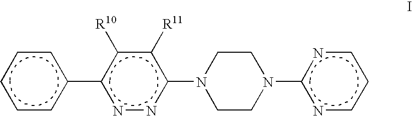
wherein R¹⁰ is hydrogen, hydroxyl, alkyl, alkoxy, alkenyl, alkynyl, alkylene, aryl, heteroaryl, sulfonyl, sulfinyl, sulfenyl, amino, imino, azido, thiol, thioalkyl, thioalkoxy, thioaryl, nitro, cyano, halo, ═O, ═S, carboxyl, carbonyl, carbamoyl, carboxamide, or phosphonate, and R¹¹ is alkyl, alkoxy, alkenyl, alkynyl, alkylene, alkenylene, alkenyloxy, aryl, aryloxy, arylalkoxy, aroyl, heteroaryl, heterocyclic, acyl, acyloxy, sulfonyl, sulfinyl, sulfenyl, amino, imino, azido, thiol, thioalkyl, thioalkoxy; thioaryl, nitro, cyano, halo, ═O, ═S, phosphonate carboxyl, carbonyl, carbamoyl, carboxamide, or ureido.

2. A stable and substantially purified synthetic pharmaceutically acceptable acid addition salt according to claim 1 wherein R¹⁰ is hydrogen, hydroxyl, alkyl, alkoxy, alkenyl, aryl, heteroaryl, sulfonyl, sulfinyl, sulfenyl, amino, thiol, thioalkyl, thioalkoxy, nitro, halo, ═O, ═S, or carboxyl; and R¹¹ is alkyl, alkenyl, alkynyl, alkylene, alkenylene, alkoxy, alkenyloxy, aryl, aryloxy, arylalkoxy, aroyl, heteroaryl.

3. A stable and substantially purified synthetic pharmaceutically acceptable acid addition salt according to claim 1 wherein R¹⁰ is hydrogen, hydroxyl, alkyl, alkoxy, sulfonyl, sulfinyl, halo, thiol, or carboxyl, and R¹¹ is alkyl, alkenyl, alkoxy, alkenyloxy, aryl, heteroaryl, amino, imino, azido, thiol, thioalkyl, thioalkoxy, nitro, cyano, halo, ═O, ═S, carboxyl, carbonyl, carbamoyl, or carboxamide; or an isomer or a pharmaceutically acceptable salt thereof.

4. A stable and substantially purified synthetic pharmaceutically acceptable acid addition salt according to claim 1 wherein R¹¹ is hydrogen, alkyl, alkoxy, amino, thiol, halo, ═O, ═S, phosphonate, or carboxyl.

5. A stable and substantially purified synthetic pharmaceutically acceptable acid addition salt according to claim 1 wherein, R¹¹ is alkyl, alkoxy, alkenyl, aryl, or heteroaryl.

6. A stable and substantially purified synthetic pharmaceutically acceptable acid addition salt according to claim 1 wherein R¹¹ is alkyl, aryl, or an unsaturated 5 to 6 membered heteromonocyclyl group containing 1 to 4 nitrogen atoms.

7. A stable and substantially purified synthetic pharmaceutically acceptable acid addition salt according to claim 1 wherein R¹¹ is alkyl or heteroaryl.

8. A stable and substantially purified synthetic pharmaceutically acceptable acid addition salt according to claim 1 wherein one of R¹⁰ and R¹¹ in a compound of the formula I is a heteroaryl.

9. A stable and substantially purified synthetic pharmaceutically acceptable acid addition salt according to claim 8 wherein the heteroaryl is an unsaturated 5 to 6 membered heteromonocyclyl group containing 1 to 4 nitrogen atoms.

10. A stable and substantially purified synthetic pharmaceutically acceptable acid addition salt according to claim 8 wherein one of R¹⁰ and R¹¹ is a pyridinyl, and the other of R¹⁰ and R¹¹ is hydrogen.

11. A stable and substantially purified synthetic pharmaceutically acceptable acid addition salt according to claim 5 wherein R¹¹ is substituted or substituted alkyl, phenyl, benzyl, or pyridinyl.

12. A stable and substantially purified synthetic pharmaceutically acceptable acid addition salt according to claim 1 wherein one of R¹⁰ and R¹¹ in a compound of the formula I is phenyl or benzyl, and the other of R¹⁰ and R¹¹ is hydrogen.

13. A stable and substantially purified synthetic pharmaceutically acceptable acid addition salt according to claim 1 wherein one of R¹⁰ and R¹¹ in a compound of the formula I is C₁-C₆ alkyl and the other of R¹⁰ and R¹¹ is hydrogen.

14. A stable and substantially purified synthetic pharmaceutically acceptable acid addition salt according to claim 1 wherein R¹¹ in a compound of the formula I is C₁-C₆ alkyl and R¹⁰ is hydrogen.

15. A stable and substantially purified synthetic pharmaceutically acceptable acid addition salt according to any preceding claim wherein the salts are halide salts.

16. A stable and substantially purified synthetic pharmaceutically acceptable acid addition salt according to any preceding claim wherein the salt is a chloride or bromide salt.

17. A stable and substantially purified synthetic pharmaceutically acceptable acid addition salt according to claim 15 or 16 wherein the counter-cation of the halide salt is an alkali metal or hydrogen.

18. A stable and substantially synthetic pharmaceutically acceptable acid addition salt according to any preceding claim wherein the salts are hydrochloride salts.

19. A stable and substantially purified synthetic pharmaceutically acceptable acid addition salt according to any preceding claim which is a di-hydrochloride salt.

20. A stable and substantially purified synthetic pharmaceutically acceptable acid addition salt according to any preceding claim which is a non-crystalline form.

21. A stable and substantially purified synthetic pharmaceutically acceptable acid addition salt according to any preceding claim which is a crystalline chloride or bromide salt of a compound of the formula I.

22. A stable and substantially purified synthetic pharmaceutically acceptable acid addition salt according to claim 1 which is a halide salt of 4-methyl-6-phenyl-3-(4-pyrimidin-2-ylpiperazin-1-yl)pyridazine.

23. A stable and substantially purified synthetic pharmaceutically acceptable acid addition salt according to claim 22 which is a chloride salt of 4-methyl-6-phenyl-3-(4-pyrimidin-2-ylpiperazin-1-yl)pyridazine.

24. A stable and substantially purified synthetic pharmaceutically acceptable acid addition salt according to claim 23 which is a hydrochloride salt of 4-methyl-6-phenyl-3-(4-pyrimidin-2-ylpiperazin-1-yl)pyridazine.

25. A stable and substantially purified synthetic pharmaceutically acceptable acid addition salt according to claim 24 which is a di-hydrochloride hydrate salt of 2-(4-(4-methyl-6-phenylpyridazin-3-yl)piperazin-1-yl)pyrimidine.

26. A stable and substantially purified synthetic pharmaceutically acceptable acid addition salt according to any preceding claim having a purity of greater than about 90%, 92%, 93%, 94%, 95%, 96%, 97%, 98%, or 99%.

27. A stable and substantially purified synthetic pharmaceutically acceptable acid addition salt according to any preceding claim having an aqueous solubility at 37° C. of about 100 to 400 mg/ml, about 100 to 350 mg/ml, about 150 to 350 mg/ml, about 200 to 350 mg/ml, or about 300 to 350 mg/ml.

28. A pharmaceutical composition comprising a substantially purified synthetic pharmaceutically acceptable acid addition salt according to any preceding claim and a pharmaceutically acceptable carrier, excipient or vehicle.

29. A method for preparing a pharmaceutical composition comprising mixing an acid addition salt of a compound of the formula I according to any preceding claim into a selected pharmaceutical carrier, excipient or vehicle, and optionally adding other therapeutic agents.

30. A method for treating a neuroinflammatory disease in a subject comprising administering an effective amount of a salt or composition according to any preceding claim.

31. Use of a salt or composition according to any preceding claim in the preparation of a medicament for treating a neuroinflammatory disease.

32. A kit comprising one or more acid addition salt of a compound of the formula I, or a composition according to any preceding claim, a container, and instructions for use.
